(19) United States
(12) Patent Application Publication
(10) Pub. No.: US 2026/0060593 A1
(43) Pub. Date: Mar. 5, 2026

(54) Integrating Three-Dimensional Medical Imaging Into Digital Electroanatomic Models

(71) Applicant: KardioNav, Inc., Fort Mills, SC (US)

(72) Inventors: Peter Michael VAN DAM, Arnhem (NL); David JENKINS, Flanders, NJ (US)

(21) Appl. No.: 19/318,666

(22) Filed: Sep. 4, 2025

Related U.S. Application Data
(60) Provisional application No. 63/691,146, filed on Sep. 5, 2024.

(51) Int. Cl. A61B 5/367; A61B 34/10; A61B 34/20; G06T 19/20; G06V 10/26; G06V 10/74; G06V 10/82

(52) U.S. Cl. CPC A61B 5/367; A61B 34/10; A61B 34/20; G06T 19/20; G06V 10/26; G06V 10/761; G06V 10/82; A61B 2034/105; G06T 2210/41; G06T 2219/2016; G06V 2201/031

(57) ABSTRACT

Various embodiments include methods for generating patient-specific heart and thorax models using anatomical landmarks and segmentation data, optimized through the application of trained neural network models. Three-dimensional medical imaging data may be processed by a trained neural network to automatically segment and isolate the heart, blood cavities, and thorax to extract feature maps from the segmented images. Anatomical landmarks, such as the heart apex and valve centers, are identified and the alignment of heart axes is verified. Reference heart and thorax models are selected and adapted to fit the patient-specific landmarks through scaling, translating, and rotating. Best adapted heart and thorax models may then be used for conducting one or more medical procedures. Neural network models, trained on historical patient data sets, may be refined through machine learning from new patient data, thereby improving accuracy.

### RELATED APPLICATIONS

This application claims the benefit of priority to U.S. Provisional Application No. 63/691,146 entitled “Integrating Three-Dimensional Medical Imaging Into Digital Electroanatomic Models,” filed Sep. 5, 2024, the entire contents of which are hereby incorporated by reference for all purposes.

### BACKGROUND

In the field of cardiac electrophysiology, three-dimensional (3D) “maps” of the timing of the electrical activation of the heart are now in medical use. Creating patient-specific models of the heart and thorax is important for various medical applications, including inverse ECG modeling. Traditional methods of reconstructing these 3D models from medical images (MRI/CT/echo) are labor-intensive and subject to subjectivity due to the required manual interventions.

In one way of generating such maps, the physician rapidly moves a catheter with electrodes around in a selected chamber of the heart. Such catheters have one or more electrodes capable of conducting minute voltages. The clearest voltage, with generally a higher amplitude, is obtained by touching the electrode to the inner surface of the chamber. That voltage, along with the timing within each electrogram of individual heartbeats, are provided as inputs to a computer. Additional data of the 3D spot on the heart measured inside a magnetic or ultrasound space is simultaneously noted with each electrode recording. As the physician obtains data from more points within the heart along what the computer sees as the outermost reach of the catheter, the computer creates a shell of the inner surface of the heart as a 3D model.

Because this 3D heart model is computer-generated based on limited input data, as well as interpolations between data points, often the resulting heart model is not accurate with respect to the actual dimensions of the chamber of the heart. Although the heart model is accurate enough for some electrophysiology work, with more advanced therapies coming forth, better accuracy of electroanatomic heart models is desired.

### SUMMARY

Various aspects include methods—and computing systems implementing embodiment methods—for generating patient-specific heart models using minimal measurements. Various aspects may enable segmenting the myocardium of a patient's heart to identify a matching digital three-dimensional (3D) heart model for purposes of mapping electrophysiology dynamics when imaging is limited to the blood pools of the endocardium.

Various aspects may include importing medical imaging data of the patient's heart and thorax into the processing system; processing the imported medical imaging data by the processing system to segment the heart, blood cavities, and thorax structures to produce segmented images; processing the segmented images by the processing system to identify anatomical landmarks, including the valves, left and right apex, and other relevant landmarks; verifying by the processing system the alignment of atrial and ventricular heart axes based on the identified anatomical landmarks; selecting by the processing system a reference heart model from a database of reference electroanatomic heart models adjusting the reference heart model by the processing system including scaling and modifying the selected reference heart model to match the patient's anatomical landmarks, and storing the resulting adapted heart model in a memory in a format suitable for use in performing a medical procedure on the patient; selecting a reference thorax model from a database of reference thorax models in a format suitable for use in performing a medical procedure on the patient; and adjusting the selected reference thorax model by the processing system including scaling and modifying the selected reference thorax model to match the patient's anatomical landmarks, and storing the resulting adapted thorax model in the memory in a format suitable for use in performing a medical procedure on the patient.

Some aspects may further include methods implemented within a computer processing system for generating and using patient-specific electroanatomic models of a patient's heart and thorax, including: importing medical imaging data of the patient's heart and thorax into the processing system; applying the imported medical imaging data to a neural network model that has been trained to receive heart and thorax medical imaging data as an input and output a patient-specific electroanatomic heart model and a thorax model; and using the patient-specific electroanatomic heart model and thorax model output from the neural network model to perform a medical procedure on the patient.

Further aspects may include a computing device having a processing system configured with processor-executable instructions to perform operations corresponding to the methods summarized above. Further aspects may include a non-transitory processor-readable storage medium having stored thereon processor-executable instructions configured to cause a processing system to perform operations corresponding to the method operations summarized above. Further aspects may include a computing device having various means for performing functions corresponding to the method operations summarized above.

### BRIEF DESCRIPTION OF THE DRAWINGS

The accompanying drawings, which are incorporated herein and constitute part of this specification, illustrate exemplary embodiments of the claims, and together with the general description given and the detailed description, serve to explain the features herein.

FIG. 1 is a schematic representation of a cardiac mapping system according to various embodiments.

FIG. 2A is a notional block diagram illustrating elements of an artificial neural network processing system.

FIGS. 2B-2C are notional block diagrams illustrating elements and methods of training a neural network using a training dataset.

FIGS. 3A-3C are process flow diagrams illustrating example methods of generating digital heart and thorax models based on patient medical imaging data in accordance with some embodiments.

FIGS. 4-7 are process flow diagrams illustrating operations that may be performed in some of the operations of the method illustrated in FIG. 3A.

FIGS. 8A-11B are illustrations of portions of a heart showing details related to the methods of various embodiments described herein.

FIGS. 12A-13B are cross-sectional medical images of portions of a patient's thorax and heart illustrating various aspects of image analysis according to some embodiments.

FIG. 14 is a component diagram of an example computing system suitable for implementing some embodiments.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the claims.

Various embodiments include importing medical imaging data and performing algorithms for creating a patient-specific heart model based on minimal measurements, potentially including generating the model with little or no medical imaging. In contrast to traditional methods that rely on extensive data and complex processing, the present system introduces a streamlined approach to 3D heart modeling that emphasizes efficiency and accessibility. By focusing on just four key anatomical landmarks—the heart apex, the Mitral valve center, the Pulmonary valve center, and the Tricuspid valve center-various embodiment methods significantly reduce the amount of input data and computational resources required to generate accurate patient-specific heart models.

This landmark-based approach simplifies the process of model adaptation by eliminating the need for comprehensive and often cumbersome segmentation of the entire heart structure. Instead, the embodiment methods leverage these four critical points to perform a series of scaling and alignment operations that morph a standard reference heart model into a highly accurate representation of the patient's heart. This not only accelerates the model generation process but also ensures that the resulting models are tailored to the unique anatomical features of each patient with minimal manual intervention.

The reduction in computational complexity achieved by the embodiment methods has significant implications for its accessibility in clinical practice. Traditional 3D heart modeling techniques often require high-powered computing systems and specialized expertise, limiting their use to well-equipped medical facilities. In contrast, the minimalist approach of the embodiment methods is designed to operate efficiently on standard computing platforms, making it accessible to a broader range of users, including those in resource-constrained environments.

This approach democratizes the use of advanced 3D heart modeling, allowing a wider spectrum of clinicians and medical institutions to adopt and benefit from this technology. Whether for routine diagnostic procedures or complex therapeutic planning, the ability to rapidly and accurately generate patient-specific heart models without the need for extensive computational infrastructure or highly specialized knowledge is a significant advancement.

The efficiency and accessibility of this landmark-based modeling approach are particularly advantageous in applications such as inverse electrocardiography (ECG) where rapid and accurate heart models are crucial for reconstructing cardiac electrical activity from body surface measurements. The streamlined process enables clinicians to quickly generate the necessary 3D models, thereby enhancing the speed and accuracy of diagnostic and therapeutic interventions.

Moreover, the simplicity of this approach allows for potential integration into a variety of other medical applications, including robotic surgery, electrophysiology studies, and personalized cardiac therapy. By lowering the barriers to entry, this innovation opens the door to broader adoption and more widespread clinical use, ultimately improving patient outcomes across a range of cardiovascular procedures.

In evaluating the efficacy of the morphing algorithm of various embodiments, extensive testing was conducted using a dataset of 94 patient-specific heart models derived from computed tomography (CT) images. The algorithm's performance was assessed by comparing the morphed models to ground truth models generated through manual segmentation by clinical experts. This comparative analysis focused on key anatomical features, particularly the endocardial volumes of the left and right ventricles.

The results of this evaluation demonstrated that the morphing algorithm of various embodiment methods exhibits a high level of accuracy, with the median percentage error for the left ventricular volume estimated at less than 13% and the right ventricular volume at less than 6%. These metrics indicate that the algorithm reliably captures the essential characteristics of the heart's ventricles, producing models that closely align with the manually segmented ground truth models. Such precision is crucial for ensuring that the patient-specific heart models are not only anatomically accurate but also clinically useful in applications such as inverse ECG modeling and other diagnostic or therapeutic procedures.

To further validate the robustness of the morphing algorithm of various embodiment methods, several statistical analyses were performed on the differences between the morphed models and the ground truth models. Key anatomical features, including the angles of the valve planes and the geometrical distances between reference points, were examined to ensure that the morphed models accurately reflected the spatial orientation and geometry of the patient's heart. The statistical analysis revealed that the median distance errors for the valve centers were within acceptable clinical limits, with values typically less than 17 mm. Additionally, the minimal distance between the points of the endocardial volumes and the valve planes showed median values of less than 2 mm, further supporting the algorithm's precision in replicating the detailed structure of the ventricles. The associated standard deviation values were also low, indicating consistent performance across the dataset and reinforcing the reliability of the algorithm in generating accurate and reproducible heart models.

The accuracy and reliability of the morphing algorithm of various embodiment methods, as evidenced by these quantitative performance metrics, underscore its potential clinical impact. Accurate patient-specific heart models are essential for a wide range of cardiovascular procedures, from planning and simulation to real-time guidance during interventions. The ability to generate these models with minimal error ensures that clinicians can rely on them for precise diagnostics and effective treatment planning.

Moreover, the consistency of the morphing algorithm of various embodiment methods across diverse patient datasets suggests that it can be effectively applied in various clinical scenarios, regardless of patient-specific anatomical variations. This versatility, combined with the algorithm's demonstrated accuracy, could make it a valuable tool for enhancing the precision of cardiac care, ultimately leading to better patient outcomes and more efficient use of medical resources.

The morphing algorithm of various embodiment methods presents a significant advancement over traditional deep-learning-based segmentation methods that are commonly used for generating three-dimensional (3D) heart models. While deep-learning techniques have demonstrated considerable success in automatic segmentation tasks, they often require large, annotated datasets and substantial computational resources to train and execute effectively. These methods typically involve complex neural network architectures that must process high-resolution 3D medical images, which can be computationally intensive and time-consuming.

In contrast, the morphing algorithm of various embodiment methods introduced is designed to achieve accurate heart model generation with a fraction of the input data and computational load. By focusing on only four key anatomical landmarks—the heart apex, Mitral valve center, Pulmonary valve center, and Tricuspid valve center—the algorithm simplifies the model adaptation process, eliminating the need for exhaustive image segmentation and the extensive training datasets required by deep-learning models. This approach not only reduces the computational complexity but also enhances the algorithm's scalability, making it more accessible for use in various clinical settings, including those with limited computational resources.

One of the key advantages of the morphing algorithm over deep-learning-based methods is its scalability. Deep-learning models often require retraining or fine-tuning when applied to different imaging modalities or when adapting to diverse patient populations due to their dependence on large, modality-specific training datasets. This retraining process can be resource-intensive and may not always yield optimal results when the available data is limited or when the model is applied to new, previously unseen types of data.

In contrast, the reliance on anatomical landmarks makes the morphing algorithm of various embodiment methods inherently more adaptable to different imaging modalities and patient anatomies without the need for extensive retraining. The algorithm's simplicity allows it to be quickly and efficiently applied to new datasets, providing accurate patient-specific models without the overhead associated with deep-learning approaches. This makes the algorithm particularly suitable for applications in clinical environments where rapid model generation is crucial and where access to extensive computational resources may be limited.

Another benefit of the morphing algorithm of various embodiment methods is its interpretability. Deep-learning models, particularly those involving deep neural networks, are often criticized for being “black boxes” with outputs that are difficult to interpret by clinicians. The decisions made by these models are based on complex patterns learned from data, which may not always be transparent or easily understood, potentially leading to issues in clinical acceptance and trust.

The morphing algorithm, on the other hand, operates on clear, interpretable anatomical landmarks and straightforward geometric transformations, making its outputs more understandable and predictable for clinicians. This transparency enhances the clinical utility of the generated heart models, as clinicians can more easily verify and validate the accuracy of the model against known anatomical features. This interpretability, combined with the algorithm's efficiency and scalability, makes it a valuable tool for a wide range of cardiovascular applications, from routine diagnostics to complex therapeutic planning.

Some embodiment may use one or more trained neural network models that are trained to perform operations of the morphing algorithm, such as segmenting the heart and related structures, identifying anatomical landmarks such as the heart apex and valve centers, checking heart axes, and/or selecting and adjusting to fit a most similar reference electroanatomic heart model for a patient's heart and thorax. Fully automated techniques may be used to segment anatomical structures without human intervention by using one or more leveraging neural network models trained using large-scale annotated training datasets produced from diverse populations and medical imaging technologies. Training such neural networks on image data annotated to identify specific features of cardiac anatomy enables the models to accurately segment and identify landmarks in medical image data, even when manual annotation may not be feasible. Various embodiments enable precise scaling, alignment, and adaptation of reference electroanatomic heart models to match patient-specific medical imaging data, thereby improving the accuracy and usability of the 3D electroanatomic heart models in medical procedures. The electroanatomic heart model produced by various aspects may then be used in a medical procedure, such as ablation therapy, electrophysiology exams, robotic surgery, etc. Additionally, machine learning methods may be utilized to continue to improve and refine the neural network models of various embodiments through continued training based on medical procedure experience and data.

Various embodiments make use of the capabilities of training one or more neural network models to automate the processing of medical image data and the modification of a selected reference digital electroanatomic heart model to match the physical features of a patient's heart. Some embodiments provide a useful approach for automatically segmenting medical imaging data into distinct anatomical structures using trained neural networks. In some embodiments, the processing system imports 3D medical imaging data from various modalities, such as MRI or CT scans, and applies a pre-processing step to enhance image quality consistency. The trained neural networks may be trained on labeled datasets of segmented images in which human experts have manually annotated specific anatomical structures using semi-automated tools or fully automated techniques like active contours, level sets, or graph-based algorithms.

Conventional methods of deriving a digital heart model adjusted to match a patient's heart involve overlaying or combining a computer-generated digital heart model with image data from a three-dimensional scan of the heart, utilizing a scanner such as a Computerized Tomography (CT) scanner or a Magnetic Resonance Image (MRI) scanner. It is generally recognized that such a 3D scan has a quite accurate image of the characteristics of the heart, including the volume of each chamber, the different structures such as the valves, the wall thickness of the heart, the vasculature around and within the heart muscle, and the different scar tissue which may be present. Two different commercial products are available today to give anatomical data on a 3D scan so the physician can more readily identify points of interest. ADAS 3D is one of the products and is offered by a Spanish company called Adas3D Medical SL, and the other is MUSIC offered by a French company called inHeart.

Another type of electroanatomic heart model may be generated using a predefined static heart model that closely resembles 3D medical images of a patient's heart, such as those generated by VIVO, which is a commercial software product marketed by Catheter Precision, Inc. This electroanatomic model is generated based on voltages obtained from electrodes on the surface of the patient's thorax rather than from an electrode catheter positioned inside the heart.

A further known method of generating an electroanatomic heart model involves using computer methods to create a 3D mesh model of heart structures based on 3D medical imaging data, and then mathematically modeling the conduction or depolarization paths, delays, and resistance through the various types of heart tissues to arrive at a model that can be used to simulate electrophysiologic behaviors. Such methods may be computer-processing intensive, and thus expensive to implement, and subject to errors that may result from inaccurate assumptions regarding different heart tissues and conduction characteristics.

While all relevant data displayed in such products provides more information for the physician user, what is lacking is an accurate overlay and matching of the different medical images of 3D scans with predefined and well-documented electroanatomic models of the heart. Although conventional solutions enable a computer to load patient 3D medical imaging scans, rarely do a patient's 3D medical images match up with a preprogrammed digital electroanatomic model of human hearts. To complete such a match-up, time-consuming editing of a preprogrammed digital electroanatomic model must be performed by an experienced technician. Even when such manual editing is performed, usually the 3D scan is shown on a computer display as a ghosting over the electroanatomic model in which the dimensions are clearly different.

Various embodiments include computing devices having processing systems configured to overcome these and other technical challenges by using one or more trained neural network models to select and then adapt a reference electroanatomic heart model from a database of such models so as to provide robust and accurate patient-specific electroanatomic heart and thorax models using anatomical landmarks and segmentation data obtained from a patient's medical imaging data. Various embodiments minimize manual interventions, reduce subjectivity, and ensure that the resulting electroanatomic heart and thorax models meet necessary mathematical boundary conditions, thereby enhancing their usability in clinical and medical procedure applications. The integration of one or more neural network/machine learning systems further refines the accuracy of the model-matching process, leveraging historical patient data to continuously improve and optimize the generated electroanatomic heart and thorax models.

By implementing mechanisms for continuous retraining and feedback, various embodiments ensure ongoing enhancement in accuracy and robustness, making it a valuable tool in medical imaging and diagnostics. Moreover, the accurate digital electroanatomic heart models produced by various embodiments enable physicians to conduct a number of medical procedures, such as ablation therapy, electrophysiology exams, robotic surgery, etc.

The term “computing device” is used herein to refer to (but not limited to) any one or all of medical system computers, workstations, servers, desktop computers, laptop computers, and other similar computing systems that include a memory for storing images and neural network computational data, and a programmable processing system that may be configured to provide the functionality of various embodiments.

The term “processing system” is used herein to refer to one or more processors, including multi-core processors, graphics processing units (GPU), neural network processing units (NPU), microprocessor units (MPU), arithmetic logic units (ALU), memory systems, etc., that are organized and configured to perform computing functions of various embodiments.

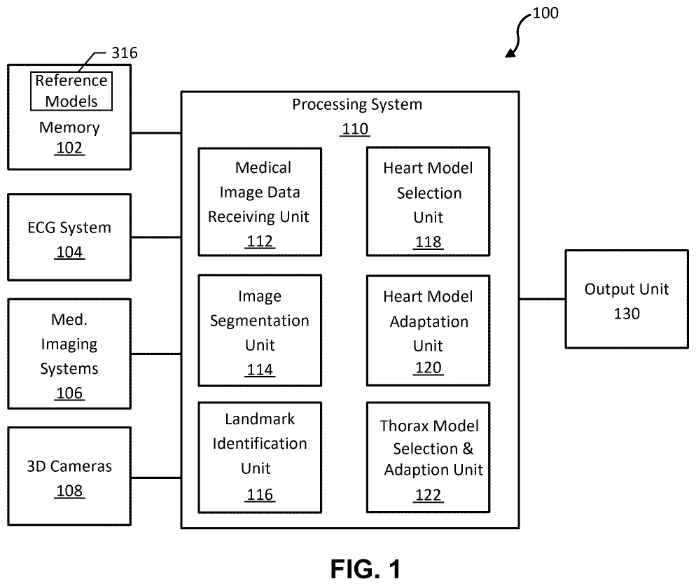

FIG. 1 is a schematic representation of a cardiac mapping system 100, according to various embodiments of the present disclosure. The system 100 may include a processing system 110, a memory 102, and an output unit 130. The memory 102 may be configured to store computer-readable data and instructions. The output unit 130 may be a monitor or other display device. The processing system 110 may be configured to execute instructions stored in the memory 102 or within the processing system 110. The processing system 110 may include a medical image data receiving unit 112, an image segmentation unit 114, a landmark identification unit 116, a heart model selection unit 118, a heart model adaptation unit 120, and a thorax model selection and adaptation unit 122. A plurality of different pre-programmed 3D electroanatomic heart models of different sizes and conditions may be saved in a reference database 316 in the memory 102.

The medical image data receiving unit 112 within the processing system 110 may be configured to receive patient data from various sources, such as an electrocardiographic (ECG) system 104, medical imaging systems 106, and/or 3D cameras 108, and may be configured to store such data in the memory 102. For example, the medical imaging systems 106 may include one or more of a magnetic resonance image (MRI) device, a computed tomography (CT) device, ultrasound imaging devices, and the like.

The processing system 110 may be configured to generate a digital electroanatomic heart model using patient-specific data generated by the ECG system 104, medical imaging systems 106, and 3D cameras 108 imported by the medical image data receiving unit 112. The image segmentation unit 114 and landmark identification unit 116 of the processing system 110 may process the 3D medical image data as described herein to identify structural landmarks and features useful for selecting and adapting a preprogrammed 3D electroanatomic heart model to conform to the patient's heart. As described herein, the heart model selection unit 118 of the processing system 110 may be configured to select from the reference model database 316 one of the 3D electroanatomic heart models having the closest conformity to the patient's heart based on the landmarks and segments identified in the medical imaging data. Then, the heart model adaptation unit 120 may adjust or modify the dimensions and other parameters of the selected predefined heart model to better match the size, shape, and other features of the patient's heart. Additionally, a predefined thorax model may be selected from the reference model database 316 and adapted to match the patient's thorax, including identifying the exterior surface in the images, by the thorax model selection and adaptation unit 122. The selected and adapted 3D electroanatomic heart model and thorax model may then be displayed or otherwise output (e.g., as data for a robotic surgical system) by the output unit 130 for use in various medical procedures.

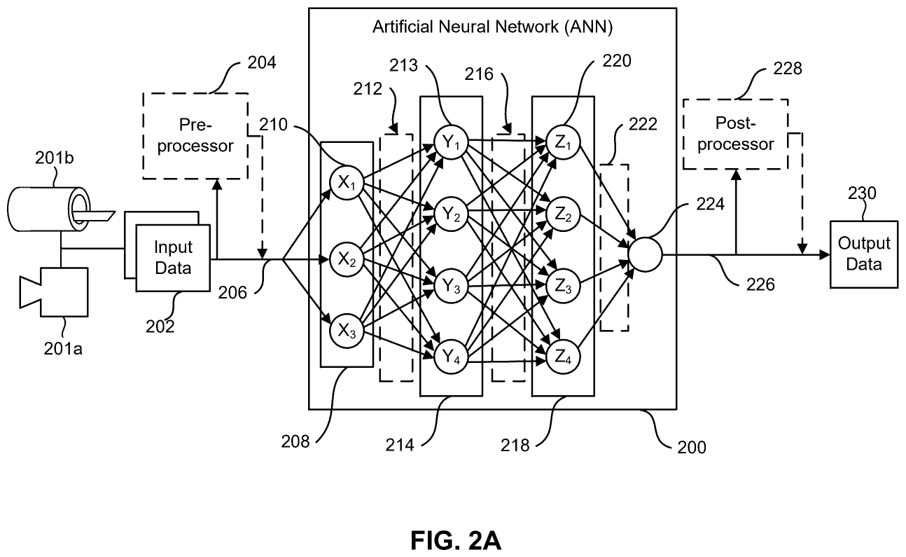

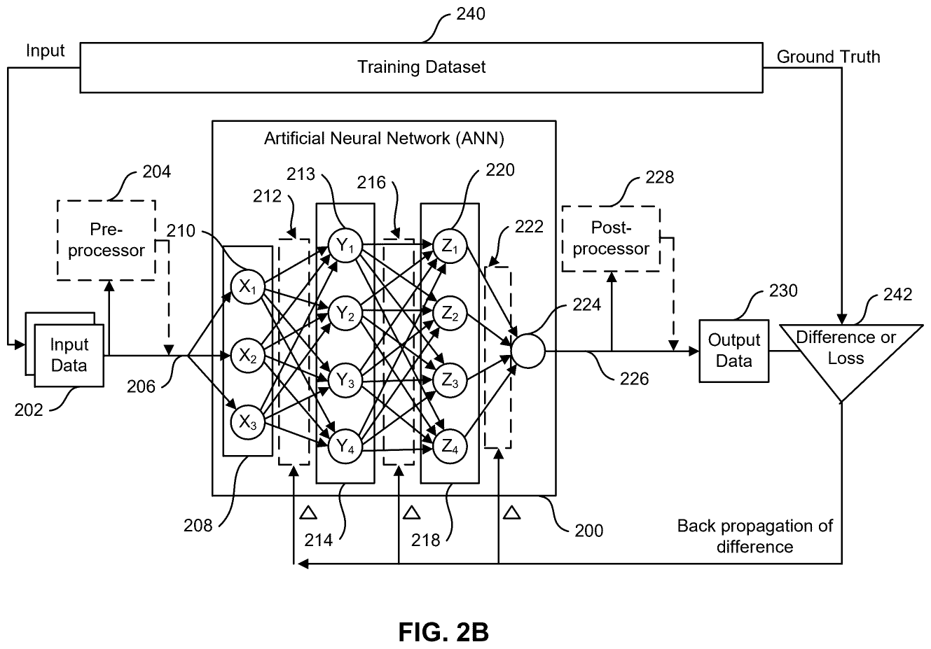

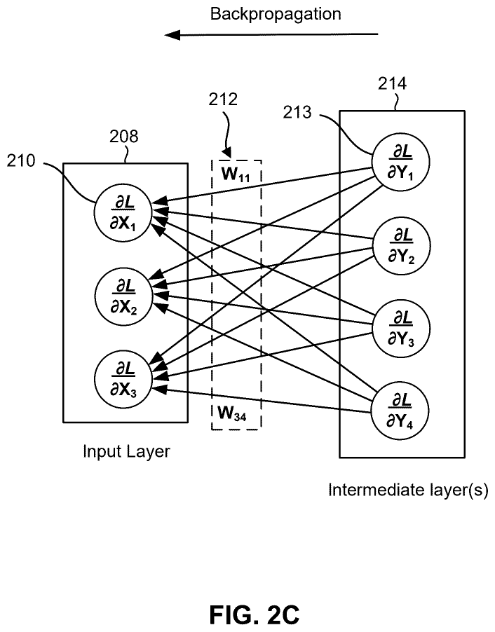

In various embodiments, some or all of the processing units 112-122 within the processing system 110 may include, make use of, or be implemented as a neural network that has been trained to receive as inputs raw or processed medical imaging data and, in the case of the heart model selection unit 118 and thorax model selection unit 122 access preprogrammed reference heart and/or thorax models 316, and output inferences regarding the inputs as described herein. Such neural network models may be similar to the example described below with reference to FIG. 2A and be trained using the methods described below with reference to FIGS. 2B and 2C.

The terms “neural network” and “neural network model” are used herein to refer to an interconnected group of processing nodes (or neuron models) that collectively operate as a software application or process that controls a function of a computing device and/or generates an overall inference result as output. Individual nodes in a neural network may attempt to emulate biological neurons by receiving input data, performing simple operations on the input data to generate output data, and passing the output data (also called “activation”) to the next node in the network. Each node may be associated with a weight value that defines or governs the relationship between input data and output data. A neural network may learn to perform new tasks over time by adjusting these weight values. In some cases, the overall structure of the neural network and/or the operations of the processing nodes do not change as the neural network learns a task. Rather, learning is accomplished during a “training” process in which the values of the weights in each layer are determined. As an example, the training process may include causing the neural network to process a task for which an expected/desired output is known, comparing the activations generated by the neural network to the expected/desired output, and determining the values of the weights in each layer based on the comparison results. After the training process is complete, the neural network may operate in an “inference” mode in which inputs (sometimes referred to as “prompts”) are processed as new tasks with the determined weights.

The term “inference” is used herein to refer to a process that is performed at runtime or during the execution of the software application program corresponding to the neural network. Inferences may be generated by inputs traversing the processing nodes in the neural network along a forward path to produce one or more values as an overall activation or overall “inference result.”

Deep neural networks implement a layered architecture in which the activation of a first layer of nodes becomes an input to a second layer of nodes, the activation of a second layer of nodes becomes an input to a third layer of nodes, and so on. As such, computations in a deep neural network may be distributed over a population of processing nodes that make up a computational chain. Deep neural networks may also include activation functions and sub-functions (e.g., a rectified linear unit that cuts off activations below zero, etc.) between the layers. The first layer of nodes of a deep neural network may be referred to as an input layer. The final layer of nodes may be referred to as an output layer. The layers in between the input and final layer may be referred to as intermediate layers, hidden layers, or black-box layers.

Each layer in a neural network may have multiple inputs, and thus multiple previous or preceding layers. Said another way, multiple layers may feed into a single layer. For ease of reference, some of the embodiments are described with reference to a single input or single preceding layer. However, it should be understood that the operations disclosed and described in this application may be applied to each of multiple inputs to a layer and multiple preceding layers.

Recurrent neural networks (RNN) are a class of neural networks particularly well-suited for sequence data processing. Unlike feedforward neural networks, RNNs may include cycles or loops within the network that allow information to persist. This enables RNNs to maintain a “memory” of previous inputs in the sequence, which may be beneficial for tasks in which temporal dynamics and the contexts in which data appear are relevant.

Long short-term memory networks (LSTM) are a type of RNN that addresses some of the limitations of basic RNNs, particularly the vanishing gradient problem. LSTMs include a more complex recurrent unit that allows for the easier flow of gradients during backpropagation. This facilitates the model's ability to learn from datasets that include large files (e.g., medical images) and remember over multiple data file inputs, making it apt for tasks such as continuous machine learning based on updates that include many large data files as may be generated and used in medical procedures and modeling of patient hearts in support of such procedures.

Many neural network models or machine learning systems use a transformer architecture, which is a specific type of neural network that includes an encoder and/or a decoder and is particularly well-suited for sequence data processing. Transformers may use multiple self-attention components to process input data in parallel rather than sequentially. The self-attention components may be configured to weigh different parts of an input sequence when producing an output sequence. Unlike solutions that focus on the relationship between elements in two different sequences, self-attention components may operate on a single input sequence. The self-attention components may compute a weighted sum of all positions in the input sequence for each position, which may allow the model to consider other parts of the sequence when encoding each element. This may offer advantages in tasks that benefit from understanding the contextual relationships between elements in a sequence, such as sentence completion, translation, and summarization. The weights may be learned during the training phase, allowing the model to focus on the most contextually relevant parts of the input for the task at hand. Transformers, with their specialized architecture for handling sequence data and their capacity for parallel computation, often serve as foundational elements in constructing large generative AI models (LXM).

Large generative AI models (LXM) are an advanced computational framework that includes any of a variety of specialized AI models including, but not limited to, large language models (LLMs), large speech models (LSMs), large/language vision models (LVMs), vision language models (VLMs)), hybrid models, and multi-modal models. An LXM may include multiple layers of neural networks (e.g., RNN, LSTM, transformer, etc.) with millions or billions of parameters. In various embodiments, LXMs may operate independently as standalone units, may be integrated into more comprehensive systems and/or into other computational units, and/or may interface with specialized hardware accelerators to improve performance metrics such as latency and throughput. In some embodiments, the LXM component may be enhanced with or configured to perform an adaptive algorithm that allows the LXM to better understand context information and perform operations in support of an overall analysis process. In some embodiments, the adaptive algorithms may be performed by the same processing system that manages the core functionality of the LXM and/or may be distributed across multiple independent processing systems.

The term “embedding layer” is used herein to refer to a specialized layer within a neural network, typically at the input stage, that transforms discrete categorical values or tokens into continuous, high-dimensional vectors. An embedding layer may operate as a lookup table in which each unique token or category is mapped to a point in a continuous vector space. The vectors may be refined during the model's training phase to encapsulate the characteristics or attributes of the tokens in a manner that is conducive to the tasks the model is configured to perform.

The term “token” is used herein to refer to a unit of information that a generative neural network may receive as a single input during training and inference. Each token may represent any of a variety of different data types. For example, in the case of medical image data, each token may correspond to a portion or patch of an image (e.g., pixel blocks), sequences of video frames, etc. In the case of ECG data, tokens may be assigned to signal values within brief time slots during heartbeat cycles or small segments of an ECG waveform. In neural networks that process medical image data, different tokens may be used to represent data in different sources of medical imagery, such as MRI, CT, and ultrasound image data. In hybrid systems that combine multiple modalities (text, speech, images, ECG waveforms or time slices, etc.), each token may be a complex data structure that encapsulates information from various sources. For example, a token may include both image and ECG information, each of which independently contributes to the token's overall representation of heart anatomy and electrophysiology that is addressed in the neural network model.

Each token may be converted into a numerical vector via the embedding layer. Each vector component (e.g., numerical value, parameter, etc.) may encode an attribute, quality, or characteristic of the original token. The vector components may be adjustable parameters that are iteratively refined during the model training phase to improve the model's performance during subsequent operational phases. The numerical vectors may be high-dimensional space vectors (e.g., containing more than 300 dimensions, etc.) in which each dimension in the vector captures a unique attribute, quality, or characteristic of the token. Such intricate representation in high-dimensional space may help the trained neural network understand the physical and electrical interaction subtleties of the training data inputs. During operational or inference usage, the tokens may be processed sequentially through layers of the trained neural network, which may include structures or networks appropriate for sequence data processing, such as transformer architectures, recurrent neural networks (RNNs), or long short-term memory networks (LSTMs).

The term “sequence data processing” is used herein to refer to techniques or technologies for handling ordered sets of tokens in a manner that preserves their original sequential relationships and captures dependencies between various elements within the sequence. The resulting output may be a probabilistic distribution or a set of probability values, each corresponding to a “possible succeeding token” in the existing sequence. For example, in image recognition and manipulation tasks, a trained neural network may suggest the possible succeeding adjacent pixel block token determined to have the highest probability in view of surrounding pixel blocks. The trained neural network may choose the token with the highest determined probability value to augment an image analysis or generation, which may subsequently be fed back into the model for further image analysis or generation.

Referring to FIG. 2A, a neural network 200 useful in various embodiments may include an input layer 208, intermediate layer(s) 214, 220, and an output layer 224. Each of the layers 208, 214, 220, 224 may include one or more processing nodes 210, 213, 220 (labeled as X₁-X₃, Y₁-Y₃, and Z₁-Z₃) that receive input values, perform computations based on the input values and weights 212, 216, 222, and propagate the result (referred to as “activations”) to the next layer (illustrated as arrows).

Artificial neurons 210, 216, 220, 224 in each layer 208, 214, 218, 224 may be activated by or be responsive to parameters, such as the weights and biases 212, 216, 222 of the neural network 200. The weights and biases of the neural network 200 are adjusted during a training process to control the strength of connections between layers 208, 214, 218, 224 or artificial neurons 210, 216, 220, 224, while the biases 212, 216, 222 control the direction of connections between the layers or artificial neurons. An activation function may select or determine whether an artificial neuron 210, 216, 220, 224 transmits its output to the next layer or not in response to its received data.

In feed-forward neural networks, such as the neural network 200 illustrated in FIG. 2A, the computations are performed as a sequence of operations on the outputs of a previous layer (e.g., 208, 214, 218). The final set of operations generates the output 224 of the neural network, such as a probability that an image contains a specific item (e.g., stop sign, yield sign, speed limit sign, etc.) or information indicating that a proposed action should be taken. Many neural networks 200 are stateless.

The neural network 200 illustrated in FIG. 2A includes fully-connected (FC) layers 208, 214, 218, 224, which are also sometimes referred to as multi-layer perceptrons (MLPs). In a fully-connected layer 208, 214, 218, 224, all outputs are connected to all inputs (illustrated by arrows). Each processing node's activation is computed as a weighted sum of all the inputs received from the previous layer based on weights 212, 216, 222.

Referring to layers 208 and 214 in FIG. 2A, an example computation performed by the processing nodes and/or neural network 200 may be: yⱼ=f(Σ³ᵢ₌₁ Wᵢⱼ*xᵢ+b), in which Wᵢⱼ are weights (illustrated as matrix 212), xᵢ is the input to the layer, yⱼ is the output activation of the layer, f(⋅) is a non-linear function, and b is bias. For example, the neural network 200 may be configured to receive as input data 202 pixels of medical images (i.e., input values) from cameras 201a, computer tomography (CT) or magnetic resonance imaging (MRI) 201b, electrocardiogram (not shown), ultrasound imaging (not shown), etc. in the first layer, and generate outputs indicating the presence of different low-level features (e.g., lines, edges, volumes, etc.) in the image. At a subsequent layer, these features may be combined to indicate the likely presence of higher-level features, such as the shape and dimensions of portions of the patient's heart and/or thorax. For example, in the training of a neural network for generating a digital electroanatomic heart and/or thorax model or intermediate results used in generating such models for various embodiments, pixels within imaging data may be combined into shapes, shapes may be combined into sets of shapes, etc., and at the output layer 224, the neural network 200 may generate a probability value that indicates whether adjustments to one or more library heart or thorax models best matches the patient's medical image data.

As illustrated in FIG. 2A, a neural network 200 may be supplemented by pre-processing of medical imaging input data 202 and post-processing of an inference output 226 of the neural network to facilitate the generation of useful output data 230 in the form of digital heart and/or thorax models. For example, medical image data from various types of medical images, such as obtained by cameras 201a, CT or MRI systems 201b, etc., may be pre-processed by a pre-processor 204, such as to crop, enhance, reformat, or otherwise transform raw medical image and other medical data into a format suitable for use as an input 206 to the first layer 208. Similarly, the output 226 of the neural network output layer 224 may be post-processed by a post-processor 228 to translate, reformat, or otherwise transform the inference into output data 230 that is usable for generating digital heart and/or thorax models based on the medical imaging input data 202.

FIG. 2B is a notional block diagram of a neural network 200 illustrating the training process. A neural network 200 for use in various embodiments may be trained on how to transform medical input data 202/306 into proper output inferences 226 for generating digital heart and/or thorax models. The overall structure of the neural network 200, and operations of the processing nodes and layers 208, 214, 218, 224 do not change as the neural network learns the task. Rather, the training process adjusts the values of the weights and biases of each layer so that the correct output inference 226 is produced for a given input 206.

Training the neural network 200 may include causing the neural network 200 to process a training or previously acquired set of medical imaging data, referred to as a training dataset, for which an expected/desired output in the form of digital heart and/or thorax models is known (such as manually generated data or input data and outputs from previous uses of the neural network. This training dataset provides the ground truth for the inference that the neural network should produce from processing the training dataset. An error or difference can be determined by comparing the output generated by the neural network 200 to the expected/desired output (i.e., ground truth). This difference between the ground truth output and the output generated by the neural network 200 is referred to as loss (L) or difference.

To train a neural network 200, the training dataset 240 is used to provide the input data 202 that is processed by the neural network. In the training process, the neural network 200 receives a set of training input data, performs calculations through the various network layers 208, 214, 218, 224 by applying biases and weights 212, 216, 222 to produce an output 226 or output data 230. The output 226 or output data 230 is compared to the ground truth associated with the corresponding input data 202 to calculate a difference or loss 242. This difference or loss is then provided in a backpropagation process to the layers and weights in the neural network to provide feedback that is used to adjust the biases and weights of the neural network so as to reduce the difference or loss.

Backpropagation may operate by passing values backward through the neural network 200 to compute how the loss is affected by each weight between each layer. The backpropagation computations may be similar to the computations used when traversing the neural network 200 in the forward direction (i.e., during inference). To improve performance, the difference or loss (L) from multiple sets of input data (“a batch”) may be collected and used in a single pass for updating the weights in the neural network. Many passes may be required to train the neural network 200 with weights suitable for use during inference operations of generating a patient-specific electroanatomic heart model based on medical image data and a selected digital heart model.

Referring to FIG. 2C, during training, the weights (wᵢⱼ) may be updated using a hill-climbing optimization process called “gradient descent.” The gradient indicates how the weights should change in order to reduce the difference or loss (L). In the backpropagation computations, various weights may be adjusted based upon the relative contribution to the difference of each weight, sometimes referred to as the gradient effect of each weight. This gradient indicates how the weights should change in order to reduce the difference or loss (L). A multiple of the gradient of the loss relative to each weight, which may be the partial derivative of the difference or loss L with respect to the weight

$$( e . g . , \frac{\partial L}{\partial X 1} , \frac{\partial L}{\partial X 2} , \frac{\partial L}{\partial X 3} ) ,$$

could be used to update the weights 212, 216, 222.

Different activation functions may be used to model different types of non-linear relationships. By introducing non-linearity into a machine learning neural network model, an activation function allows the configuration of the machine learning neural network model to change in response to identifying or detecting complex patterns and relationships in the input data 202. Some non-limiting activation functions include a sigmoid-based activation function, a hyperbolic tangent (tanh) based activation function, a convolutional activation function, up-sampling, pooling, and a rectified linear unit (ReLU) based activation function.

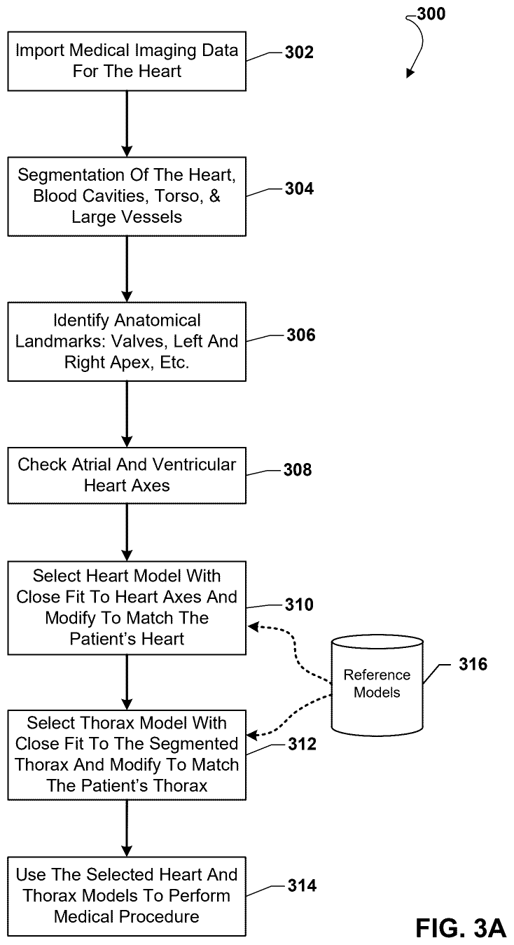

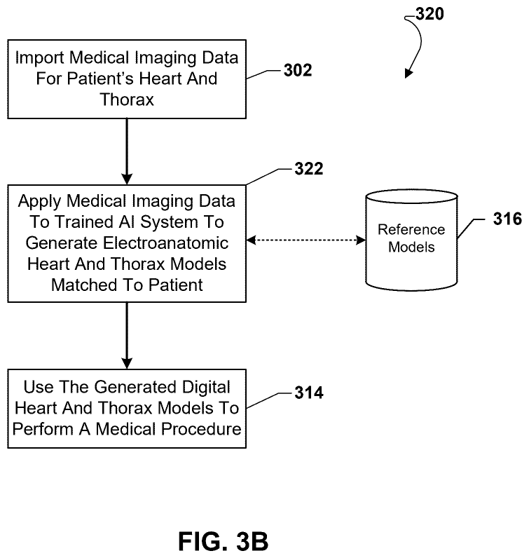

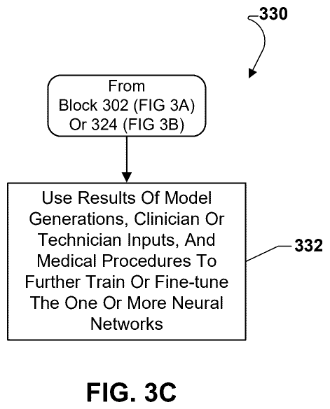

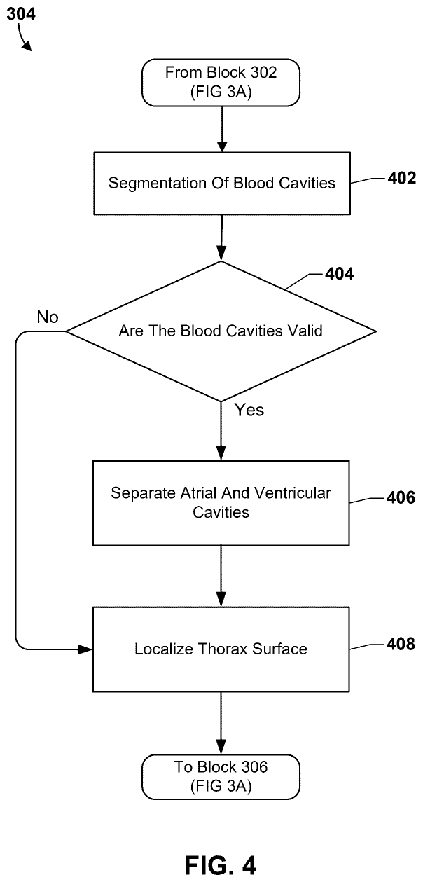

Various embodiments make use of neural network training methods as described above to generate one or more neural network models that are trained to receive patient medical imaging and other data as inputs and then output inference results that either support the generation of electroanatomic heart and/or thorax models according to the method 300 described below with reference to FIG. 3A or output inference results in the form of a generated electroanatomic heart and/or thorax model according to the method 320 described below with reference to FIG. 3B. The neural network training methods may also be used to refine, retrain, and/or fine-tune the one or more trained neural network models based on subsequent patient medical imaging data and procedure results, as described below with reference to FIG. 3C.

In some embodiments, the processing system performing training of one or more neural networks may include temporal information in the training through the incorporation of time-series data in the training datasets. This allows models to learn time-dependent patterns in anatomical changes. Such training enables neural network models to provide inference outputs based on both spatial and temporal cues during feature extraction and probability map generation.

In some embodiments, the processing system performing training of one or more neural networks may incorporate multimodal data streams from multiple sensors (e.g., MRI images, CT images, ECG recordings, positron emission/computed tomography (PET) scans, ultrasound images, photographic images, etc.) into training datasets for enhanced robustness against variations in patient-specific anatomy or imaging modalities. This approach enables neural network models to learn generalizable features that integrate information across different sensing channels while maintaining overall accuracy on unseen cases.

In some embodiments, the processing system performing training of one or more neural networks incorporates reinforcement learning techniques during model adaptation by providing rewards based on performance metrics such as precision and recall for specific anatomical structures. This allows neural network models to adapt over time through continuous feedback mechanisms in response to changing patterns within diverse populations or clinical contexts. By leveraging both supervised and unsupervised training paradigms, neural network models may learn generalizable features that balance accuracy with robustness against variations due to patient-specific morphology.

In some embodiments, the processing system may incorporate transfer learning from pre-trained networks on large datasets for enhanced performance in specific anatomical structures (e.g., cardiac chambers). For example, the processing system may utilize transfer learning by pre-training a neural network model on large datasets of medical images from diverse modalities before fine-tuning it using domain-specific data. This approach enables leveraging knowledge learned from general anatomical structures to adapt quickly to specific clinical applications or patient populations with varying characteristics. Leveraging both domain expertise and machine learning algorithms during neural network model training may facilitate adapting to changing clinical contexts and technologies (e.g., the introduction of new medical testing and imaging technologies and formats).

In some embodiments, the processing system performing training of one or more neural networks may employ spatial pyramid pooling (SPP) for aggregating features across multiple scales and orientations.

In some embodiments, one or more trained neural network models are used to automate specific image processing and matching processes within an overall computerized method of generating and using a patient-specific electroanatomic heart model, as described with reference to FIG. 3A. In some embodiments, a patient-specific electroanatomic heart model may be generated by a neural network model that is trained to receive medical image data as an input and output a matched electroanatomic heart model, as described with reference to FIG. 3B. In some embodiments, one or more neural network models may be refined, fine-tuned, or otherwise continuously improved using machine learning techniques based on medical image data, technician or physician inputs, and results of medical procedures using the patient-specific electroanatomic heart models generated by various embodiments.

FIG. 3A illustrates a method 300 of generating and using a patient-specific electroanatomic heart and thorax model based on medical image data in accordance with some embodiments. With reference to FIGS. 1-3A, the method 300 may be performed in a computing device by a processing system 110 and other components or subsystems discussed in this application. Means for performing the functions of the operations in the method 300 may include a processing system 110 and other components described herein. Further, one or more processors of a processing system may be configured with software or firmware to perform some or all of the operations of the method 300. In order to encompass the alternative configurations enabled in various embodiments, the hardware implementing any or all of the method 300 is referred to herein as a “processing system.”

In block 302, the processing system may be configured to perform operations including importing medical imaging data into the computer system. The imported imaging data may be provided or configured as the input for subsequent segmentation and model adaptation processes. The medical imaging data typically includes Digital Imaging and Communications in Medicine (DICOM) files from CT, MRI, PET, ultrasound, single-photon emission computerized tomography (SPECT), and other imaging scans, which provide detailed 3D images of the patient's heart and thorax. The medical imaging data obtained in block 302 may further include ECG data, as well as timing information (e.g., metadata) correlating medical imaging data with ECG data.

The operations in block 302 may further include preprocessing techniques to enhance image quality and consistency. In some embodiments, spatial filters such as Gaussian blur or median filtering may be applied to reduce noise artifacts; frequency-domain filters like the Fourier transform may also be used for denoising purposes. Wavelet-based methods may be employed in some cases where high-frequency components need to be removed while preserving low-frequency information. Also, in some embodiments, in cases in which patients have undergone multiple scans at different institutions using varying modalities (e.g., MRI, CT, PET, ultrasound, etc.), the processing system may integrate these disparate data sets into a unified 3D model by applying proprietary algorithms that normalize pixel intensities across devices.

In some embodiments, image filtering or enhancement may be accomplished by applying the medical image data to a trained neural network that has been trained on a training data set of annotated medical images to enhance features in medical images at multiple levels of abstraction and generate high-fidelity image data. Such a neural network model may be similar to the example described with reference to FIG. 2A and be trained using methods described with reference to FIGS. 2B and 2C. As described above, such a neural network may employ convolutional layers configured to process medical images, with each layer capturing distinct patterns such as edges, textures, and complex shapes.

In block 304, the processing system may be configured to segment the heart, blood cavities, and thorax structures within the medical imaging data to produce segmented images. Initially, automatic segmentation of the heart, blood cavities, and thorax may be performed. The segmentation process identifies and separates the left and right blood cavities, which are then validated to ensure accuracy. This segmentation defines the anatomical boundaries necessary for further analysis and model adaptation.

The operations in block 304 may include creating segmentation masks from the probability maps by assigning each pixel to the anatomical structure with the highest likelihood. The segmentation masks may then be created by assigning the most probable structure to each pixel based on the probability maps, resulting in a clear, labeled image delineating different anatomical features. Additionally, feature extraction from the segmented images using a trained neural network may be employed, with max pooling layers used to reduce the spatial dimensions of the feature maps while retaining the most salient features.

In some embodiments, graph-cut algorithms may be employed for segmenting overlapping structures such as blood vessels. This approach involves defining a weighted undirected graph where nodes represent pixels in an image and edges connect adjacent pixels with weights reflecting their similarity based on intensity values from the 3D medical scan. The algorithm then finds a minimum cut or maximum flow between two sets of vertices to separate regions within the thorax region.

In addition, level set methods may be used for segmenting anatomical structures by iteratively evolving an initial contour through image data until it reaches its desired position based on energy minimization principles and boundary conditions, on which a neural network model may be trained based on information in the training dataset provided by expert annotations. This approach may be particularly useful when dealing with complex boundaries or irregular shapes such as those found in cardiac anatomy.

In some embodiments, the processing system may use active contours for segmenting complex boundaries between overlapping regions within the thoracic region. This approach involves initializing a contour around an initial estimate of the boundary and iteratively updating it using energy-based optimization techniques to minimize the difference between the estimated segmentation mask and ground truth annotations provided by experts or semi-automated tools.

In some embodiments, automatic segmentation may be accomplished in block 304 by the processing system applying the medical image data to a neural network that has been trained on a training data set of annotated medical images to extract features from the medical images at multiple levels of abstraction and generate high-dimensional feature maps. For example, the neural network may be trained to process medical image data received in block 302 and output the information required for performing the operations in block 306 as described next. Such a neural network model may be similar to the example described with reference to FIG. 2A and be trained using methods described with reference to FIGS. 2B and 2C. As described above, such a neural network may employ convolutional layers configured to process medical images, with each layer capturing distinct patterns such as edges, textures, and complex shapes.

In some embodiments, the neural network may include max pooling layers configured to reduce the spatial dimensions of the feature maps, retaining the most critical features while decreasing the computational load. The feature maps may be processed through a series of fully connected layers that learn non-linear relationships between anatomical structures and their corresponding probability distributions to capture edges, textures, and complex shapes within segmented images. The neural network may output a set of high-dimensional feature maps that represent the input images, highlighting the structures and segments of interest.

The neural network may output may be in the form of probability maps and segmentation masks for the heart, blood cavities, and thorax. Each probability map may indicate the likelihood that each pixel in the segmented images belongs to specific anatomical structures. A probability map assigns a value between 0 and 1 to each pixel, reflecting the likelihood that the pixel belongs to a particular anatomical structure.

In some embodiments, probability maps may be generated using hierarchical attention networks (HANs) that selectively focus on relevant features and regions of interest during feature extraction. This approach improves the handling of overlapping structures or anatomical variations by allowing the model to concentrate efforts where the most informative cues reside. HAN-based architectures may be used to generate detailed segmentations from coarse-grained annotations.

In some embodiments, the processing system may incorporate domain knowledge through the use of expert labels and manual annotation tools during training of the neural network model. This enhances adaptation of the neural network to specific clinical contexts or anatomical structures while maintaining overall robustness against variations in patient-specific morphology. By leveraging both labeled data and unlabeled images for pre-training, the models learn generalizable features that adapt across diverse populations.

The segmentation process may involve multiple layers of 3D convolutions followed by max pooling operations to capture different patterns such as edges, textures, and complex shapes in the medical images. In some embodiments, spatial filters (e.g., a Gaussian filter) may be applied before input to the neural network, such as noise reduction or frequency-domain filtering techniques like the Fourier transform.

In block 306, the processing system may be configured to perform operations including identifying key anatomical landmarks, including the heart apex and valve centers, from the segmentation masks. In some embodiments, the operations in block 306 may be performed automatically by the processing system, such as using a trained neural network model. However, identifying anatomical landmarks may be performed manually, checked manually, or identified partially automatically and partially manually (e.g., manually identifying anatomical landmarks that the processing system is unable to identify due to image artifacts or missing data). In the automatic mode, the processing system may use segmentation data to determine the plane defined by the mitral valve, the tricuspid valve, and the left ventricular apex by fitting a 3D ellipse to the left ventricular cavity. The manual mode serves as an alternative when automatic identification fails, allowing for manual localization of valves and apex. The identified landmarks, including the valve planes and their perpendicular directions, provide reference points for scaling and aligning the reference heart model to the patient's anatomical heart structures.

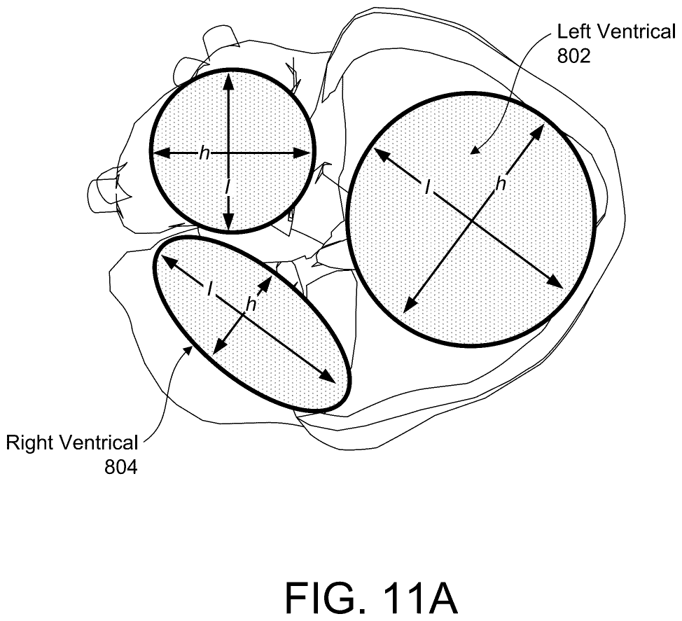

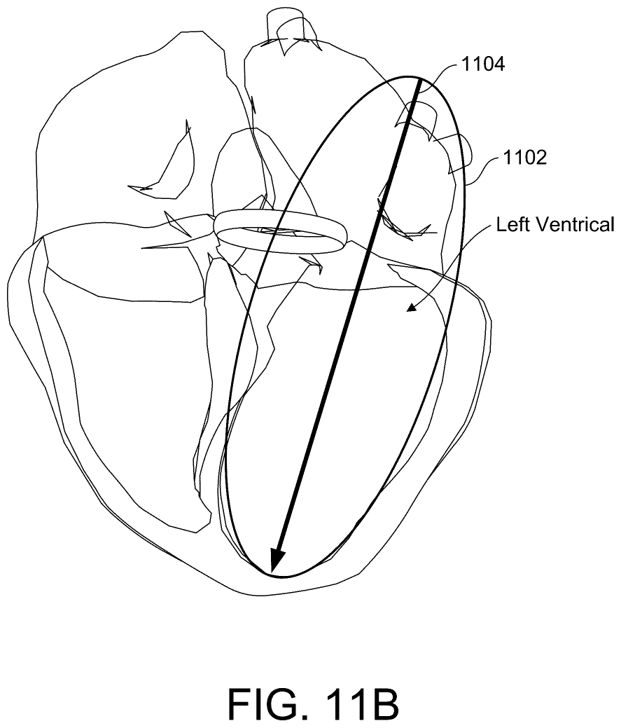

In some embodiments, identifying key anatomical landmarks in block 306 may include fitting a 3D ellipse to the left ventricular cavity to determine the heart apex as illustrated in FIG. 11B.

In some embodiments, the processing system may identify spatial relationships between anatomically relevant structures using graph-based methods to identify patterns within segmented images. This approach enables better handling of overlapping or adjacent regions by modeling their interdependencies.

In some embodiments, some or all of the processes in block 306 may be performed by applying the medical imaging data to a neural network model that has been trained to process such image data and output the information required for performing the operations in block 308 as described next. Such a neural network model may be similar to the example described with reference to FIG. 2A and be trained using methods described with reference to FIGS. 2B and 2C.

In block 308, the processing system may be configured to perform operations including checking atrial and ventricular heart axes. These operations may include verifying the alignment of atrial and ventricular heart axes using the identified anatomical landmarks. Ensuring the consistency of the heart axes is part of verifying the alignment of the identified landmarks. The directions of the valve planes may be checked to confirm they point toward the left ventricular apex. If any discrepancies are found, the direction of the plane may be inverted to maintain consistency. These operations may provide data that will enable a reference heart model to be accurately aligned with the patient's anatomical features, which is necessary for the subsequent adaptation and fitting processes.

In some embodiments, verifying the alignment of atrial and ventricular heart axes using the identified anatomical landmarks in block 308 may include checking the directions of the valve planes to confirm they point toward the left ventricular apex and inverting the direction of any inconsistent planes.

In some embodiments, some or all of the processes in block 308 described above may be performed by applying the medical imaging data to a neural network model that has been trained to process such data and output the information required for performing the operations in block 310 as described next. Such a neural network model may be similar to the example described with reference to FIG. 2A and be trained using methods described with reference to FIGS. 2B and 2C.

In block 310, the processing system may be configured to perform operations including selecting from a database 314 of reference electroanatomic heart models the best reference heart model that closely fits the patient's heart axes as determined from the medical imaging data. Initially, a reference heart model with pre-determined anatomical landmarks may be loaded from the database 314. This reference heart may be scaled and adapted to match the patient's heart parameters using the identified landmarks identified in block 306. Adapting the loaded reference heart model may include adapting the model to fit the segmented heart by scaling and translating the reference heart model to match up to the landmarks and segments of the patient's heart model. As part of the operations in block 310 or as a further operation, the adapted heart model may be compared to the patient's heart landmarks to ensure accuracy. If the adapted model provides a better fit, it is stored as the new patient-specific heart model. This process may be repeated for other reference heart models obtained from the reference model database 316 until the best-fitting model is identified. This process enables identifying an adapted reference electroanatomic heart model that provides the best fit and accuracy in representing the patient's heart.

In block 312, the processing system may be configured to perform operations including selecting from the reference model database 314 a reference thorax model that best fits the patient's thorax. In some embodiments, a reference thorax model may be loaded from the database, translated, and scaled to align with the patient's thorax. This alignment process may minimize the distance between the segmentation points and the thorax model. Rotation of thorax imaging data is generally not required as patients are typically in a standard supine position during MRI, CT, and other scans. The processing system may compare the adapted thorax model to the patient's anatomical landmarks, and if the adapted model provides a better fit to the patient's thorax than previously evaluated models, it is stored as the new patient-specific thorax model. This process may be repeated for other reference thorax models in the reference model database 316 until an adapted thorax model that best fits the patient's thorax imagery is selected.

In some embodiments, the processing system may use active contours within the medical imaging data for segmenting complex boundaries between overlapping regions within the thoracic region. This approach involves the processing system initializing a contour around an initial estimate of the boundary and iteratively updating the contour using energy-based optimization techniques to minimize the difference between the estimated segmentation mask. In some embodiments, this process may be supplemented by manual annotations of medical imaging data provided by experts or semi-automated tools.

In block 314, the processing system may be configured to perform operations, including using the generated electroanatomic heart and thorax models to perform one or more medical procedures. For example, the generated electroanatomic heart model may be used during an ablation therapy procedure to guide the clinician to an arrhythmia initiation site or conduction branch where ablation may be applied. As another example, the generated heart and thorax models may be used as inputs to a computer-aided or robotic surgery system to facilitate accurate navigation of tools within the patient to reach a location or conduct a surgical procedure. As another example, the generated electroanatomic heart model may be used by a clinician inserting a pacemaker lead into the patient's heart to identify a suitable pacing location and then attach the lead at that location.

In a further application, comparing medical images and heart landmark measurements to a large library of heart models, anatomical defects may be recognized based on minimal medical images and measurements. Comparison of heart measurements to a library of deviating hearts may be used to classify heart shapes, such as size (large or small), dilation, valve sizes and shapes, etc. Alternatively, comparisons of heart measurements to a library of normal hearts may enable recognition of deviant hearts based on out-of-normal-range scaling and rotation factors. In some embodiments, a trained neural network model may be used to interpret heart measurements and parameters for such purposes.

FIG. 3B illustrates a method 320 of generating and using a patient-specific electroanatomic heart model based on medical image data input to a neural network model trained to receive such data and output digital heart and thorax models in accordance with some embodiments. With reference to FIGS. 1-3B, the method 320 may be performed in a computing device by a processing system 110 implementing the trained neural network model. Means for performing the functions of the operations in the method 320 may include a processing system 110 and other components described herein. In order to encompass the alternative configurations enabled in various embodiments, the hardware implementing any or all of the method 320 is referred to herein as a “processing system.

As described with reference to FIG. 3A, in block 302, the processing system may perform operations including importing medical imaging data into the computer system, such as DICOM files from CT, MRI, or other medical imaging scans that provide 3D imagery of the patient's heart and thorax, as described for the like-numbered block in the method 300.

In block 322, the processing system may be configured to perform operations including applying the imported medical imaging data (e.g., as an input or prompt) to a neural network model that has been trained to generate electroanatomic models of the patient's heart and thorax matched to the patient's heart and thorax based on the medical imaging data. The neural network executed in block 322 may be trained using a training database that includes input files of patient medical imaging data and corresponding electroanatomic models of the heart and thorax that were obtained from the reference models database 316. This training dataset may be developed by accumulating the results of many applications of the method 300 described with reference to FIG. 3A, and organizing each patient's medical image data in the form of a prompt input correlated to the corresponding best-fit adapted electroanatomic models of the heart and thorax as ground truth results that can be used in backpropagation training of the neural network, as described with reference to FIGS. 2B and 2C.

In some embodiments, the trained neural network used in block 322 may be supplemented with algorithms that obtain information from a reference model database 316 as part of operations leading to the generation of electroanatomic models of the patient's heart and thorax. For example, the processing system may be configured to pre-process the medical imaging data received in block 302 to classify or size the patient's heart and/or thorax, and use that information to select a reference model of the heart and/or thorax from the reference database 316, and then generate a prompt for the trained neural network that includes both the medical imaging data and the selected model(s) as inputs. In such embodiments, the neural network may be trained to receive both medical imaging data and a reference heart and/or thorax model as inputs and generate patient-specific heart and thorax electroanatomic models. In some embodiments, the pre-processing to classify and select a suitable input heart and/or thorax reference model(s) may be performed by a separate neural network that has been trained to make such a selection.

In some embodiments, by training a neural network with such a training dataset, the neural network may learn how to generate accurate electroanatomic models of the heart and thorax based on a patient's medical imaging data without reference to a database of reference heart and thorax models (e.g., 316). This is because the knowledge included in the reference heart and thorax models may be inherently incorporated, effectively encoded, into the training dataset through the ground truth patient-specific electroanatomic models of the heart and thorax. Thus, in such embodiments, the reference model database 316 may not be necessary or used once such a neural network has been trained, except perhaps for retraining and updating fine-tuning.

In block 314, the processing system may be configured to perform operations including using the generated electroanatomic heart and thorax models to perform one or more medical procedures as described for the like-numbered block in the method 300.

FIG. 3C illustrates a method 330 of refining, fine-tuning, or otherwise continuously training a neural network model for generating patient-specific electroanatomic heart and thorax models based on medical image data input to a neural network model trained to receive such data and output digital heart and thorax models in accordance with some embodiments. With reference to FIGS. 1-3B, the method 320 may be performed in a computing device by a processing system 110 implementing the trained neural network model. Means for performing the functions of the operations in the method 320 may include a processing system 110 and other components described herein. In order to encompass the alternative configurations enabled in various embodiments, the hardware implementing any or all of the method 320 is referred to herein as a “processing system.

In block 332, the processing system may be configured to perform operations including implementing a machine learning system to refine the accuracy of the model matching process by continuously learning from historical patient data sets, including 3D scans, electro-anatomical models, and ECG recordings, and the corresponding adapted best-fitting heart and thorax models. In these operations, the processing system may use results of model generations, clinician or technician inputs, and medical procedures to further train or fine-tune one or more neural networks used in either the method 300 or the method 320. As described with reference to FIGS. 2B and 2C, such training, retraining, or fine-tuning of neural networks can be accomplished by using the generating patient-specific electroanatomic heart and thorax models as ground truths for corresponding patient medical imaging data to support backpropagation training of the neural network(s). In some embodiments, the processing system may periodically retrain the machine learning model with new patient data to continuously improve its accuracy and robustness. In this way, as the number of patient datasets increases, the neural network model(s) can identify better matches of medical images to electroanatomic models to provide more accurate models.

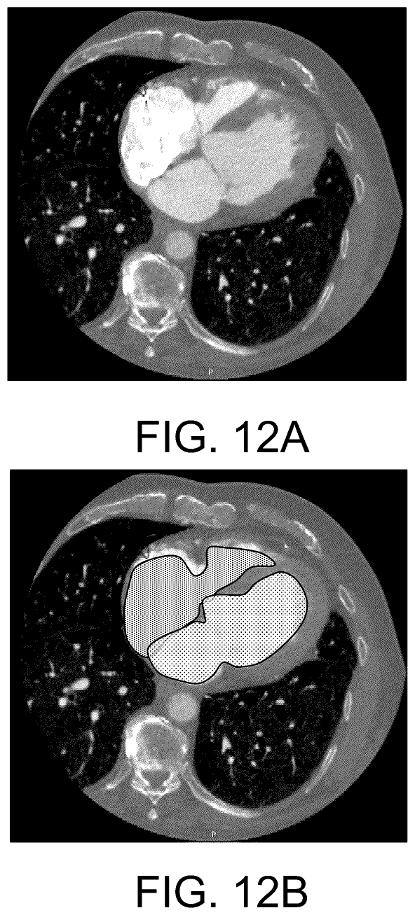

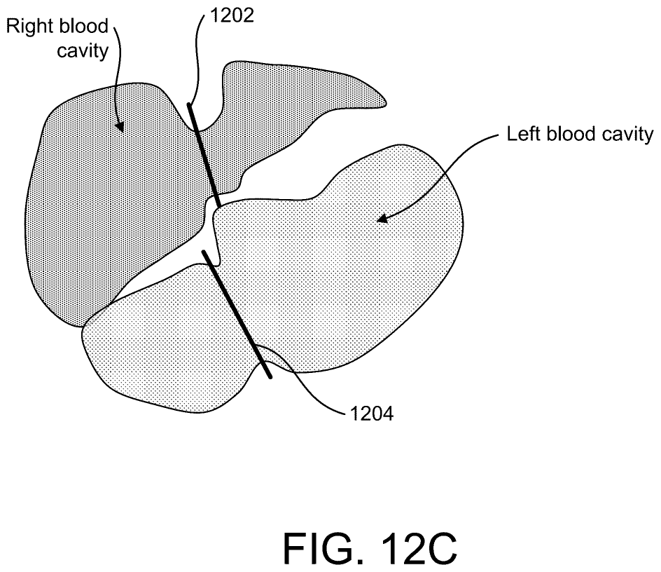

FIG. 4 illustrates operations that may be performed as part of the operations in block 304 of the method 300 (FIG. 3A) in some embodiments. With reference to FIGS. 1-4, after receiving medical imaging data inputs in block 302, the processing system may perform operations of segmenting blood cavities in block 402. The processing system may employ automatic algorithms that analyze medical images to segment out blood cavities. An example of such imaging and the resulting segmentation of blood cavities in the heart is illustrated in FIGS. 12A through 12C. In some embodiments, an automatic algorithm may identify structural elements of a patient's heart based on contrasts and boundaries between light and dark portions of the images, and then identify and record locations and coordinates of voids outlined by heart structures within the heart that correspond to blood cavities.

In some embodiments, as part of the operations in block 402, the processing system may apply the medical image data as an input to a trained neural network that has been trained on a training dataset of labeled medical image data to identify and output extracted features of blood cavities. As described, a trained neural network model may include convolutional layers that are configured and trained to capture information regarding different patterns in medical image data, such as edges, textures, and complex shapes.

In some embodiments, the trained neural network used in block 402 may be configured and trained to produce probability maps and segmentation masks for the heart that identify the blood cavities. A probability map is an intermediate output in which each pixel has a value between 0 and 1, indicating the likelihood that the pixel belongs to a specific anatomical structure, such as a blood cavity of the heart. A segmentation mask may be created by such a neural network trained to assign the most likely structure to each pixel based on the probability maps, resulting in a clear, labeled image that delineates different anatomical features, including blood cavities.

In some embodiments, a trained neural network used by the processing system may include max pooling layers configured to reduce the spatial dimensions of feature maps, retaining the most important features recognized in medical images while reducing computational load. The output of such a trained neural network may be a set of high-dimensional feature maps that identify the blood cavities in the heart.

In determination block 404, the processing system may be configured to perform operations, including determining whether the blood cavities identified in block 404 are valid. In some embodiments, the processing system may compare the identified blood cavities to preprogrammed dimensional rules, ratios, valid shape boundaries to which blood cavities should conform for the identified cavities to be valid, i.e., consistent with expected or normal range dimensions, shapes, orientations, and locations relative to recognized anatomical features (e.g., heart and/or thorax structures). Identified blood cavities that do not conform to expected or normal range structural features may be determined to be invalid.

In some embodiments, the processing system may perform the operations in determination block 404 by applying the medical image data as an input to a neural network that has been trained on a training dataset of medical image data of blood cavity segmentation images labeled as valid and/or invalid to recognize valid and/or invalid blood cavities and output a validity determination.

In response to determining that the identified blood cavities are valid (i.e., determination block 404=“Yes”), the processing system may be configured to perform operations including further processing the segmented blood cavity image portions to separate the atrial and ventricular cavities and identifying the associated valves (i.e., aortic valve, mitral valve, tricuspid valve and pulmonary valve) in block 406. In some embodiments, the processing system may distinguish the atrial and ventricular cavities based on the shapes and orientations of the segmented blood cavities. The processing system may then identify and localize the valves by identifying the constrictions in the blood cavities, such as shown by the lines 1202 and 1204 illustrated in FIG. 12C.

In some embodiments, the processing system may identify the locations of the valves by applying the segmented blood cavity results from block 402 to a neural network model that has been trained on a dataset of such image data to recognize and localize the heart valves.

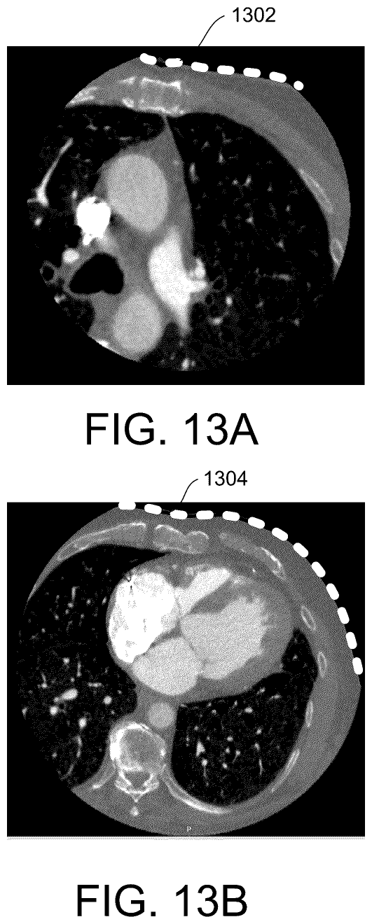

After separating the atrial and ventricular cavities and identifying the associated valves in block 406 or in response to determining that the identified blood cavities are not valid (i.e., determination block 404=“No”), the processing system may be configured to perform operations including localizing surfaces of the patient's thorax in the medical image data in block 408. In some embodiments, these operations may include the processing system using image analysis tools to identify the surface of the thorax that is visible to the clinician (i.e., the outside surface) based on positions in the imagery where there is a transition from the background (e.g., black pixels) to thorax structures (e.g., lighter pixels). This identification of the thorax surface boundary is illustrated by the dashed lines 1302, 1304 in FIGS. 13A and 13B.

In some embodiments, the processing system may perform the operations in determination block 408 by applying the medical image data as an input to a neural network that has been trained on a training dataset of medical image data of thorax images labeled to identify the thorax surface and output an indication of the thorax surface in the data.

With the operations in blocks 402-408 performed, the processing system may perform the operations in block 306 of the method 300 as described above.

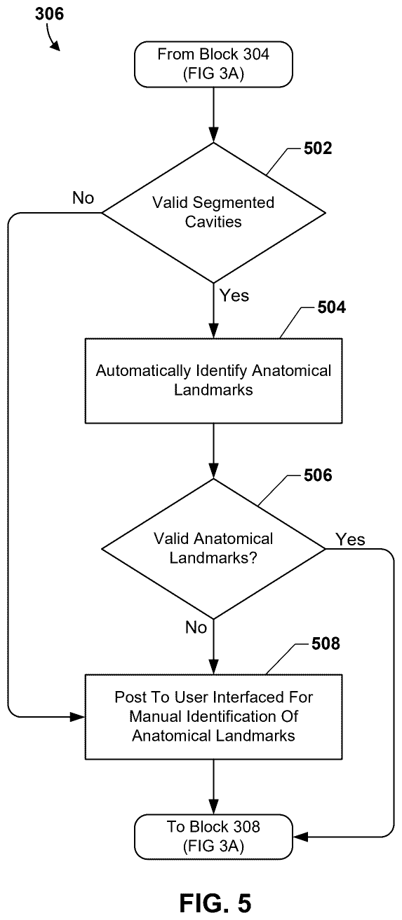

FIG. 5 illustrates operations that may be performed as part of the operations in block 306 of the method 300 (FIG. 3A) for identifying key anatomical landmarks, including the valves, and the left and right apex. With reference to FIGS. 1-5, after performing the segmentation operations on medical imaging data inputs in block 304 or blocks 402-408, the processing system may identify anatomical landmarks in the heart, either automatically if the segmented heart cavities are valid, or manually via a user interface system otherwise.

In various embodiments, the generation of patient-specific three-dimensional (3D) heart models leverages a minimalist yet highly effective approach by focusing on four critical anatomical landmarks: the heart apex, the center of the Mitral valve, the center of the Pulmonary valve, and the center of the Tricuspid valve. These four landmarks serve as the foundation for a novel morphing algorithm, which significantly simplifies the process of constructing accurate 3D heart models with minimal input data.

The use of these specific landmarks is useful for defining the geometric structure of the heart. By identifying and utilizing just these four points, the methods and computing system are capable of scaling and aligning a reference heart model to match the unique anatomical features of a patient's heart. This approach not only reduces the computational complexity typically associated with creating 3D heart models but also minimizes the need for extensive manual interventions, thereby reducing subjectivity and potential errors in the modeling process.

By focusing on these four anatomical landmarks, the methods and computing system may implement a streamlined approach to 3D heart modeling that is both efficient and accessible. This methodology contrasts with traditional modeling techniques that require comprehensive anatomical data and complex segmentation processes. The simplicity of the landmark-based approach makes it feasible for broader adoption across various clinical settings, including those with limited computational resources or specialized expertise. Consequently, various embodiments open up new possibilities for the rapid and accurate generation of patient-specific heart models, which may enable advanced medical procedures and diagnostics.

In determination block 502, the processing system may evaluate the results or output of the segmentation operations in block 304 or 402 to determine whether the segmented cavities are valid. A number of automatic segmentation algorithms exist that may be used to complete at least some of the operations in determination block 502, especially for the left ventricle. Examples include: ADAS 3D offered by ADAS 3D Medical, S.L. of Barcelona, Spain; inHEART MODELS offered by inHEART Medical of Bordeaux, France, and the 3D Slicer open source software platform. Such algorithms use regional growing algorithms in combination with AI to complete heart image segmentation. However, such algorithms may not produce valid results when working with poor contrast imaging that exhibits limited distinction between the endocardium and epicardial fat and the myocardium. These determinations in determination block 502 may involve comparing each of the blood cavity segmentations against databases or rules defining valid blood cavity shapes, sizes, and locations in human hearts to identify any unusual or inconsistent blood cavity shapes, sizes, or locations. In some embodiments, the operations in determination block 502 may be replaced by or supplemented by the evaluations performed in determination block 404 as described above with reference to FIG. 4.

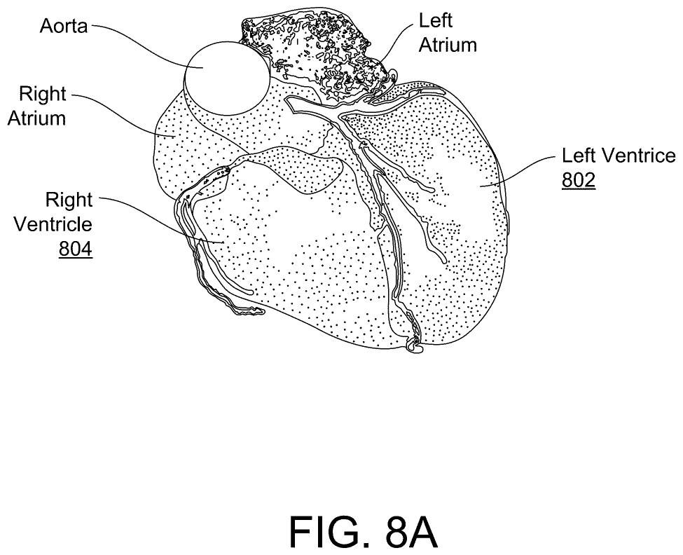

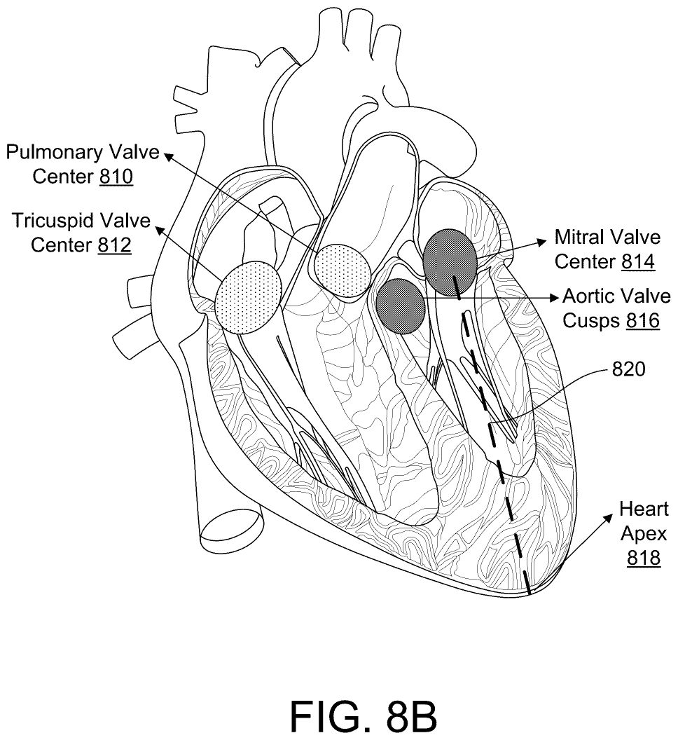

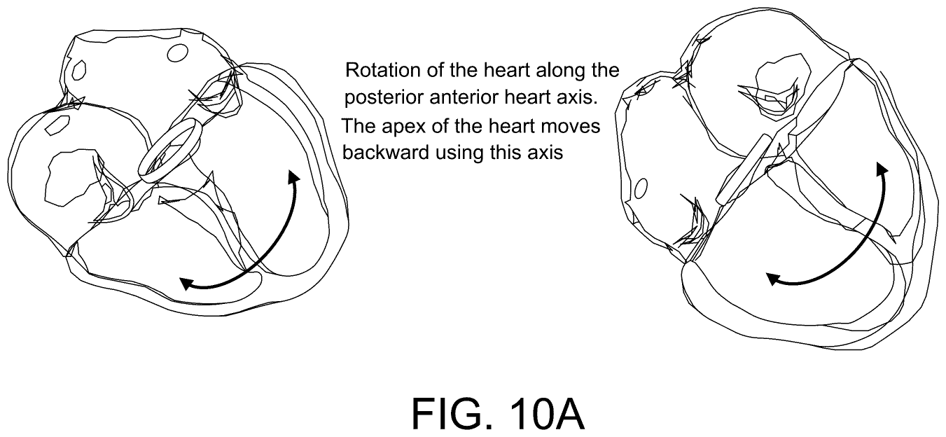

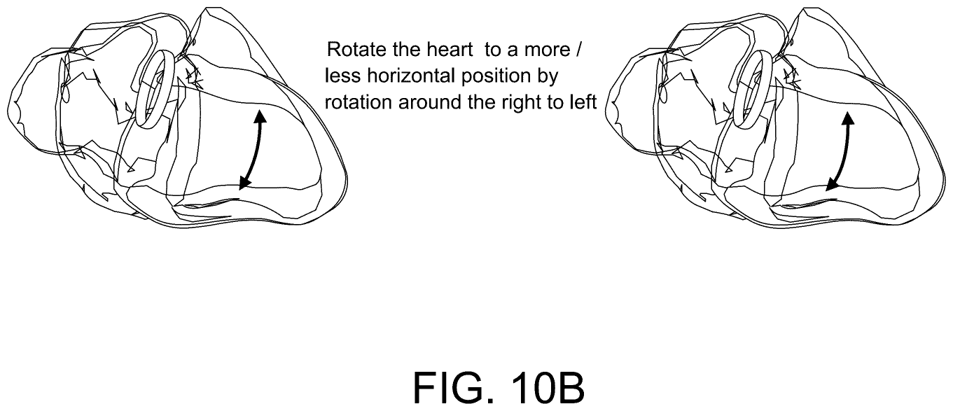

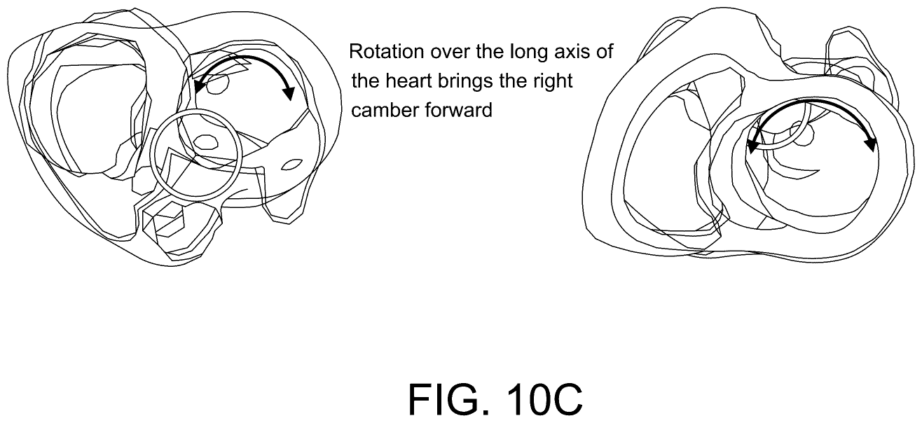

In response to determining that the blood cavity segmentations are valid (i.e., determination block 502=“Yes”), the processing system may be configured to perform operations including automatically identifying anatomical landmarks of the patient's heart using the blood cavity segmentations and structures in block 504. In some embodiments, these operations may include the processing system using image processing techniques to identify structural elements within the medical images of the heart and identify specific structural landmarks (e.g., illustrated in FIG. 8B) based on location, shape, and orientation within the heart. From the manual or automatic identified structures (e.g., valves, apex, etc.), the centers may be automatically determined to identify the landmarks. As part of the operations involved in identifying heart landmarks in block 504, the processing system may perform image rotation operations to manipulate the medical imagery to obtain cross-sectional views of different parts of the heart, as illustrated in FIGS. 10A-10C. Such processing may output the locations (e.g., coordinate values within medical image data) and identity of the identified heart landmarks.

In some embodiments, the processing system may perform the operations in block 504 by applying the medical image data as an input to a neural network that has been trained on a training dataset of medical image data of heart images labeled to identify landmarks that are useful for characterizing the patient's heart suitable for the remaining operations in the method 300.

In determination block 506, the processing system may be configured to perform operations including determining whether the heart landmarks identified in block 504 are valid. In some embodiments, the processing system may compare each of the identified landmarks against databases or rules regarding valid size, orientation, and relationship of heart structures to identify any unusual or inconsistent landmark locations, sizes, or orientations, i.e., inconsistent with expected or normal range dimensions, shapes, orientations, and locations. Identified heart landmarks that do not conform to expected or normal range landmarks may be determined to be invalid.

In some embodiments, the processing system may perform the operations in determination block 506 by applying the medical image data and identified landmarks as inputs to a neural network that has been trained on a training dataset of medical image data and heart landmarks labeled as valid and/or invalid to recognize valid and/or invalid landmark and output a validity determination.

In response to determining that some segmented cavities are not valid (i.e., determination block 502=“No”) or that one or more identified landmarks are not valid (i.e., determination block 506=“No”), the processing system may be configured to perform operations including posting to a user interface some or all of the medical imaging data with user interface tools to enable an expert (e.g., a cardiologist or radiologist) to manually identify blood cavities and/or anatomical landmarks in block 508. Some embodiments may include specialized software tools tailored specifically for medical imaging applications on the user interface that enable an expert to manually add or correct labeling annotations. The option for manual identification of heart landmarks ensures that even in situations with limited image quality, the resulting 3D models remain accurate representations of the patient's anatomy. In some embodiments, semi-automated tools may be employed to facilitate manual annotation, such as by providing interactive visualization capabilities for reviewing image data and correcting landmarks and labeling through iterative refinement processes. Such tools may also be used to receive expert annotations of medical imaging data to generate initial training datasets that include high-quality ground truth annotations useful for training neural network models, as discussed with reference to FIGS. 2B and 2C.

In response to determining that the anatomical landmarks are valid (i.e., determination block 506=“Yes”) or after receiving expert inputs identifying anatomical landmarks in block 508, the processing system may perform the operations in block 308 of the method 300 as described.

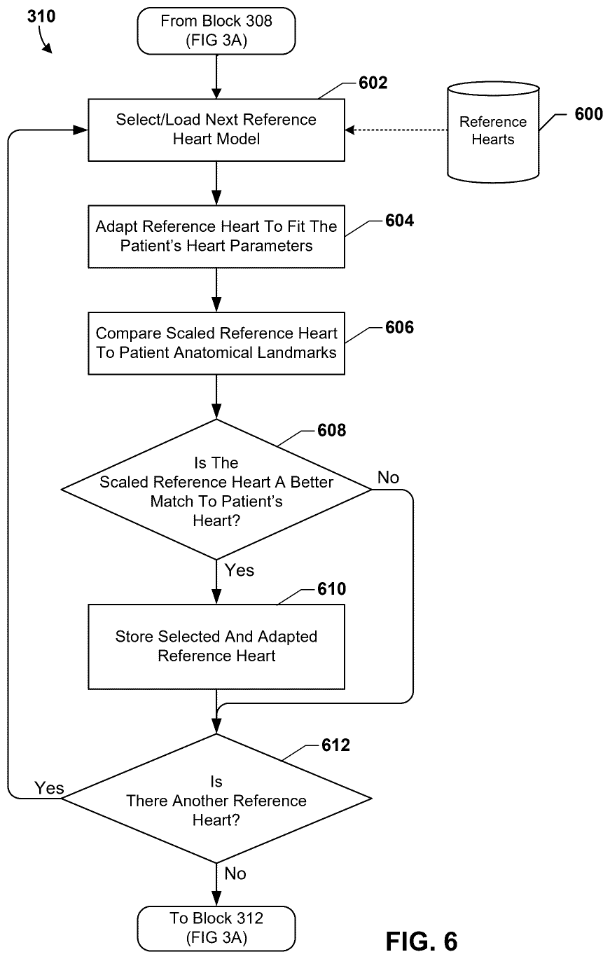

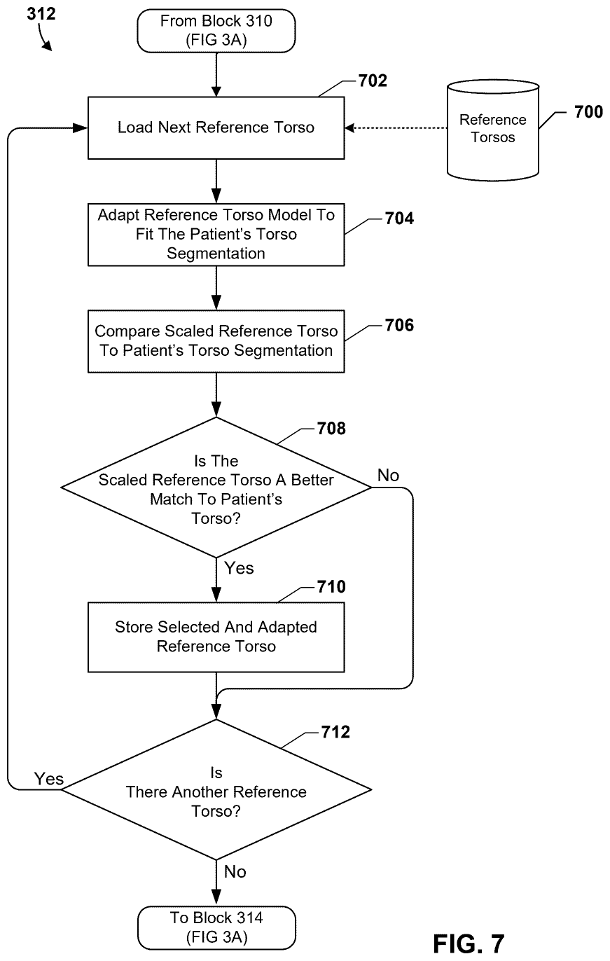

FIG. 6 illustrates operations that may be performed as part of the operations in block 310 of the method 300 (FIG. 3A) in some embodiments. With reference to FIGS. 1-6, after performing the operations in blocks 304-308, the processing system may perform operations of selecting from a database 600 a reference electroanatomic heart model for adaptation and comparison to the patient's heart.

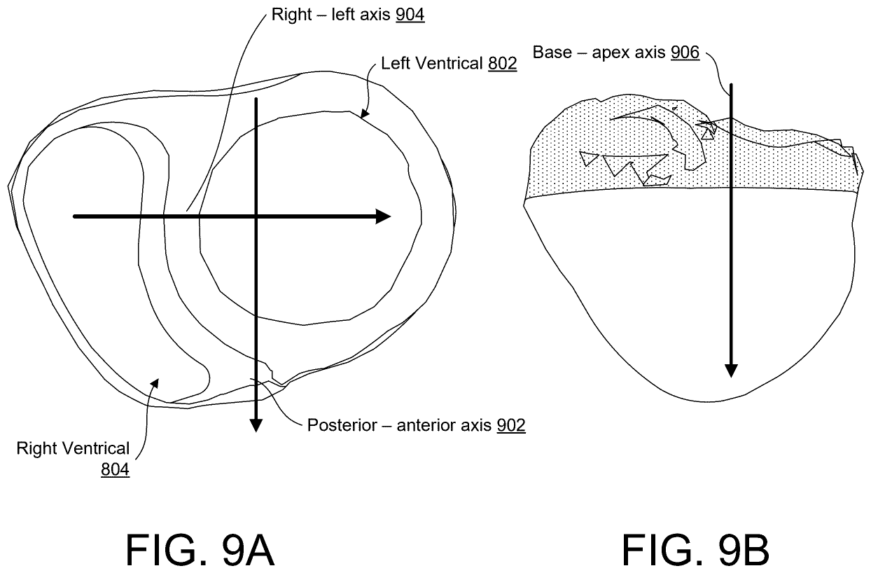

In block 604, the processing system may be configured to perform operations including adapting or “morphing” the selected reference electroanatomic heart model to fit the patient-specific anatomical landmarks by scaling, translating, and rotating the reference heart model to align with the patient's heart based on the results of segmentation and landmark identification performed in blocks 304-306. The operations in block 604 that may be performed by the processing system using an image manipulation or morphing algorithm to adapt the selected reference electroanatomic heart model to fit the patient's heart parameters (i.e., segmentation and identified anatomical landmarks). Such an algorithm may include the following scaling and translation processes, which are described with reference to the structures, axes, and rotations illustrated in FIGS. 8A-9B. Illustrations of the effects of various rotations about three different axes of a reference heart model are illustrated in FIGS. 10A-10C.

In various embodiments, the patient-specific heart modeling system may employ a novel morphing algorithm designed to transform a standard reference heart model into a highly accurate representation of a patient's heart by using minimal yet essential anatomical data. This algorithm may focus on the heart's ventricles and utilizes the four key anatomical landmarks—the heart apex, the Mitral valve center, the Pulmonary valve center, and the Tricuspid valve center—to guide the morphing process.

The morphing algorithm may operate in two primary stages: scaling and alignment. Initially, the algorithm may scale the heart model along the cardiac apex-base axis, the left-right axis, and the posterior-anterior axis based on the spatial relationships of the identified landmarks. This scaling may be driven by the ratios between the landmark distances in the patient's anatomical data and those in the reference model, ensuring that the overall dimensions of the ventricles are accurately matched to the patient's heart. This scaling ensures that the overall dimensions of the reference model conform to the patient-specific measurements derived from the identified anatomical landmarks.

Following the scaling process, the morphing algorithm performs an alignment process that further refines the accuracy of the adapted heart model with respect to the patient. This alignment involves translating the scaled heart model so that the heart apex of the reference model aligns with the corresponding apex in the patient's data. Next, the morphing algorithm rotates the heart model to align the vectors defined by the heart apex and the mitral valve center, and then fine-tunes the alignment using the projections of the pulmonary valve center onto a defined plane.

These alignment steps ensure that the spatial orientation of the heart's ventricles in the reference model matches that of the patient's actual heart. This process minimizes errors that could arise from anatomical variations, making the resulting heart model highly accurate and reliable for medical applications such as inverse electrocardiography (ECG) and other diagnostic or therapeutic procedures.

The morphing algorithm may begin with the alignment of the heart model by translating and rotating it to match the precise locations of the heart apex, mitral valve center, pulmonary valve center, and tricuspid valve center as identified in the patient's imaging data. The alignment process ensures that the reference model accurately reflects the spatial orientation and geometry of the patient's heart, thus enhancing the model's utility in clinical applications such as inverse ECG modeling and other electrophysiological procedures.

The processing system may perform scaling by using three scaling factors determined by the length of the heart axes from the reference heart model and the patient's heart. Specifically, the processing system may perform scaling along the cardiac apex-base axis 906. The processing system may perform scaling along the cardiac left-right axis 904. The processing system may perform scaling along the cardiac posterior dash anterior axis 902. In performing these scaling operations, the ratio of the distance between the heart apex 818 and the mitral valve center 814 may be used as an Apex/Base (A/B) factor that can be used to scale the heart in the apex base direction. The processing system may maintain a ratio of the distances between the mitral valve center 814 and the tricuspid valve center 812 in the patient's heart image data and the selected reference model, using a left/right (L/R) factor. Finally, the valve planes of the patient's heart and the selected reference model may be established by using the heart apex 818, mitral valve center 814, and tricuspid valve center 812 identified in the patient's heart image data and the selected reference heart model, respectively. Then, the processing system may project the pulmonary valve center 810 in the patient's heart image data and the selected reference model into planes. The ratio of the distance between the pulmonary valve center 810 and its projection in the patient's heart image data and the selected reference model may be used as a Posterior/Anterior (P/A) factor. By determining these three scaling factors (i.e., A/B, L/R, and P/A), the processing system can scale the heart model in three major (i.e., perpendicular) axes of the heart with different scaling factors.

The processing system may align the scaled selected reference heart model to the patient's heart based on feature segmentation and the defined anatomical landmarks by translating and rotating the scaled heart model until various axes and landmark vectors line up.

In a translation process, the processing system may translate (i.e., move within a reference frame defined by the processing system) the scaled reference heart model until the heart apex 818 in that model aligns with the heart apex 818 landmark in the patient's heart.

In a first alignment process, the processing system may identify the valves in both the scaled heart model and the patient's heart based on the segmentation and landmarks identified by the processing system (e.g., in method 300). Using the identified heart valves, the processing system may determine or identify a vector 820 defined by the line between the heart apex 818 and the mitral valve center 814 of the scaled reference heart model and a vector defined by the line between the heart apex 818 and the mitral valve center 814 of the patient's heart. The processing system may determine (e.g., compute) the angle between these two vectors on the scaled and translated heart model and the patient's heart in the current orientation. The processing system may then rotate the scaled and translated heart model around the heart apex 818 so that the two vectors of the respective heart apex-to-mitral valve center 820 are aligned (i.e., the angle between the two vectors is approximately zero degrees/radians). Next, the projections of the pulmonary valve center 810 on the scaled and translated heart model and the patient's heart on the plane defined by the heart apex 818 and the heart apex-to-mitral valve center vector 820 are computed. Finally, the processing system may compute the angle between the two projections, and the scaled/translated/rotated heart model is rotated about the heart apex-to-mitral valve center vector 820 (i.e., the heart apex-to-mitral valve center vector 820 forms the rotation axis) to reduce that angle to approximately zero degrees/radians.

In a second alternative alignment process, the processing system may use heart cavity dimensions in addition to the valve locations in the alignment calculations. In this alternative alignment process, the processing system may analyze the shapes of the cavities in the heart model and the patient's heart based on the segmentation and landmarks identified by the processing system (e.g., in method 300) to identify the left and right ventricular cavities. The left ventricle 802 has a somewhat circular shape, while the right ventricle 804 has an approximate half-moon shape as illustrated in FIGS. 9A and 11A, which provide criteria that the processing system can use for distinguishing the two cavities. The shapes of the two cavities may then be used by the processing system to determine angles for the left-to-right alignment and anterior-to-posterior alignment between the scaled heart model and the patient's heart imagery. The alignment of the heart apex-to-mitral valve center vector 820 can be accomplished using the methods described in the first process above, based on the locations of the valves. Additionally or alternatively, the processing system may define or fit an ellipse 1102, as illustrated in FIG. 11B, to the left ventricles in the heart model and patient's heart, and use the long axis 1104 of that ellipse as vectors in the heart model and patient's heart that are aligned by rotating the scaled/translated/rotated model until the vectors align.

The scaled, translated, and doubly rotated digital heart model resulting from either alignment process represents the closest alignment of the adjusted or “morphed” selected electroanatomic heart model to the patient's heart. In block 606, the processing system may be configured to perform operations including comparing the scaled, translated, and rotated (i.e., adapted heart model) reference heart model to the anatomical landmarks of the patient's heart to determine the degree to which the adapted heart model matches the patient's heart. For example, the processing system may determine distances between the same landmark points on the adapted heart model and the patient's heart landmarks when the two are aligned and calculate a metric or measure of the difference, such as totaling all differences, determining a root mean square difference, or calculating an average difference.

In determination block 608, the processing system may determine whether the currently selected and adapted heart model is a better match to the patient's heart based on the comparison in block 606 than a previously selected and adapted reference heart model (if not the first time executing the method). For example, the processing system may determine whether the metric or measure of difference calculated in block 606 is better (such as a smaller value) than the metric or measure of difference calculated in block 606 for a previously evaluated heart model.

In response to determining that the currently selected and adapted heart model is a better match to the patient's heart based on the comparison in block 606 than a previously selected and adapted heart model (i.e., determination block 608=“Yes”), the processing system may store the selected and adapted reference heart model on block 610.

In response to determining that the currently selected and adapted heart model is not a better match to the patient's heart based on the comparison in block 606 than a previously selected adapted heart model (i.e., determination block 608=“No”) or after storing the currently selected and adapted heart model in block 610, the processing system may determine whether there are any more reference heart models in the reference heart database 600 to evaluate in block 612. If so, the processing system may select another heart model from the database of reference heart models 600 in block 602 and repeat the processes in blocks 604-612. If not (i.e., all reference heart models in the database 600 have been evaluated), the processing system may perform the operations in block 312 of the method 300 as described.

The operations of the morphing algorithm described with reference to FIG. 6 offer several advantages over traditional methods of heart model generation. By focusing on a small set of critical anatomical landmarks and employing efficient scaling and alignment techniques, the algorithm may reduce the computational load and time required to generate accurate 3D heart models. Additionally, this approach minimizes the need for extensive manual input and complex segmentation processes, which are often time-consuming and prone to variability.

FIG. 7 illustrates operations that may be performed as part of the operations in block 312 of the method 300 (FIG. 3A) in some embodiments. With reference to FIGS. 1-7, after or in parallel with the selection and adaptation of a best electroanatomic heart model in blocks 310 and 312, the processing system may perform operations of segmenting blood cavities in block 702.

In block 704, the processing system may be configured to perform operations including adapting the selected reference thorax model to fit the patient-specific anatomical landmarks by scaling, translating, and rotating the reference thorax model to align with the patient's thorax based on the results of segmentation and landmark identification performed in blocks 304-306. With reference to FIGS. 1-7, the operations in block 704 may be performed by the processing system using an image manipulation algorithm to adapt the selected reference thorax model to fit the patient's thorax parameters (i.e., segmentation and identified anatomical landmarks), including scaling and translation processes similar to the operations in block 604 of the method 310 described with reference to FIG. 6. Examples of medical imaging data of a thorax are illustrated in FIGS. 12A-13B.

The scaled, translated, and doubly rotated digital thorax model resulting from either alignment process represents the closest alignment of the adjusted or “morphed” selected thorax model to the patient's thorax. In block 706, the processing system may be configured to perform operations including comparing the scaled, translated, and rotated (i.e., adapted thorax model) reference thorax model to the anatomical landmarks of the patient's thorax to determine the degree to which the adapted thorax model matches the patient's thorax. For example, the processing system may determine distances between the same landmark points on the adapted thorax model and the patient's thorax landmarks when the two are aligned and calculate a metric or measure of difference (e.g., totaling all differences, determining a root mean square difference, or calculating an average difference).

In determination block 708, the processing system may determine whether the currently selected and adapted thorax model is a better match to the patient's thorax based on the comparison in block 706 than a previously selected adapted thorax model (if not the first time executing the method). For example, the processing system may determine whether the metric or measure of difference calculated in block 706 is better (such as a smaller value) than the metric or measure of difference calculated in block 706 for a previously evaluated thorax model.

In response to determining that the currently selected and adapted thorax model is a better match to the patient's thorax based on the comparison in block 706 than a previously selected adapted thorax model (i.e., determination block 708=“Yes”), the processing system may store the selected and adapted reference thorax model in block 710.

In response to determining that the currently selected and adapted thorax model is not a better match to the patient's thorax based on the comparison in block 706 than a previously selected adapted thorax model (i.e., determination block 708=“No”) or after storing the currently selected and adapted thorax model in block 710, the processing system may determine whether there are any more reference thorax models in the reference thorax database 700 to evaluate in block 712. If so, the processing system may select another thorax model in block 702 and repeat the processes in blocks 704-712. If not (i.e., all reference thorax models in the database 700 have been evaluated), the processing system may perform operations in block 314 of the method 300 to perform one or more medical procedures using the adapted electroanatomic heart model and the adapted thorax model as described.

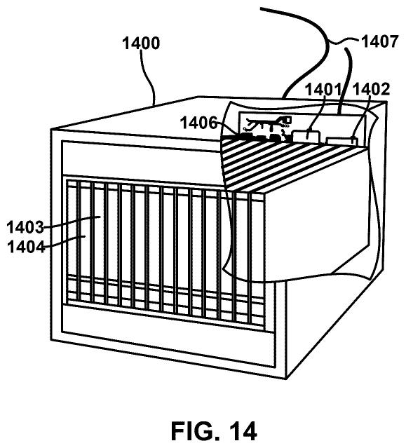

Some embodiments may be implemented on a variety of commercially available computing devices, such as the server computing device 1400 illustrated in FIG. 14. The server device 1400 may include a multi-core processor 1401 coupled to volatile memory 1402, such as RAM, and a large capacity nonvolatile memory, such as a solid-state drive 1403. The server device 1400 may also include additional storage interfaces such as universal serial bus (USB) ports and NVMe slots coupled to the processing system 1401. The server device 1400 may include network access ports 1406 coupled to the processing system 1401, enabling data connections through a network interface card 1404 and a communication network 1407 (e.g., an Internet Protocol (IP) network) connected to other network elements.

The processing systems discussed in this application may include any programmable microprocessor, microcomputer, or multiple processor chip or chips that can be configured by software instructions (applications) to perform a variety of functions, including the functions of various embodiments described. In some computing devices, multiple processors may be provided, such as one processor within a first circuitry dedicated to wireless communication functions and one processor within a second circuitry dedicated to running other applications. Software applications may be stored in the memory before they are accessed and loaded into the processor. The processors may include internal memory sufficient to store the application software instructions.

Implementation examples are described in the following paragraphs. While some of the following implementation examples are described in terms of example methods, further example implementations may include: the example methods discussed in the following paragraphs implemented by a computing device including a processor configured (e.g., with processor-executable instructions) to perform operations of the methods of the following implementation examples; the example methods discussed in the following paragraphs implemented by a computing device including means for performing functions of the methods of the following implementation examples; and the example methods discussed in the following paragraphs may be implemented as a non-transitory processor-readable storage medium having stored thereon processor-executable instructions configured to cause a processor of a computing device to perform the operations of the methods of the following implementation examples.

Further details regarding various embodiments are disclosed in the attached Appendix, which is intended to be part of the disclosure of the provisional application as if included within numbered paragraphs.
Example 1. A method implemented within a computer processing system for generating and using patient-specific electroanatomic models of a patient's heart and thorax, including: importing medical imaging data of the patient's heart and thorax into the processing system; processing the imported medical imaging data by the processing system to segment the heart, blood cavities, and thorax structures to produce segmented images; processing the segmented images by the processing system to identify anatomical landmarks, including the valves, left and right apex, and other relevant landmarks; verifying by the processing system the alignment of atrial and ventricular heart axes based on the identified anatomical landmarks; selecting by the processing system a reference heart model from a database of reference electroanatomic heart models adjusting the reference heart model by the processing system including scaling and modifying the selected reference heart model to match the patient's anatomical landmarks, and storing the resulting adapted heart model in a memory in a format suitable for use in performing a medical procedure on the patient; selecting a reference thorax model from a database of reference thorax models in a format suitable for use in performing a medical procedure on the patient; and adjusting the selected reference thorax model by the processing system including scaling and modifying the selected reference thorax model to match the patient's anatomical landmarks, and storing the resulting adapted thorax model in the memory in a format suitable for use in performing a medical procedure on the patient.
Example 2. The method of example 1, further including preprocessing the medical imaging data of the patient's heart and thorax to enhance image quality and consistency using spatial filters, frequency-domain filters, or wavelet-based methods.
Example 3. The method of either of examples 1 or 2, in which processing the imported medical imaging data by the processing system to segment the heart, blood cavities, and thorax structures to produce segmented images includes: generating probability maps by assigning pixels in the medical imaging data to anatomical structures with a highest likelihood of corresponding to such structures; and creating segmentation masks based on the generated probability maps.
Example 4. The method of any of examples 1-3, in which processing the segmented images by the processing system to identify anatomical landmarks includes the processing system using a trained neural network model to automatically identify the anatomical landmarks.
Example 5. The method of any of examples 1-4, further including: repeating operations of: selecting another reference heart model from the database of reference heart models if there is a reference heart model in the database that has not already been selected; adapting the selected heart model to match the patient's anatomical landmarks in the memory; comparing a similarity of the resulting heart model to the patient's heart medical image data with a similarity of a previous adapted heart model saved in the memory; and storing the resulting heart model in the memory if the resulting heart model is more similar to the patient's heart medical image data than the previous adapted heart model saved in the memory; and using the heart model and thorax model stored in the memory to perform a medical procedure on the patient.
Example 6. The method of example 5, in which comparing the similarity of the resulting heart model to the patient's heart medical image data with the similarity of a previous adapted heart model saved in the memory includes comparing measures of dimensional similarity of the resulting and previous adapted reference heart models to the patient's anatomical landmarks.
Example 7. The method of example 5, in which the medical procedure includes using the heart model and thorax model stored in the memory during an ablation therapy procedure to guide a clinician to an arrhythmia initiation site or conduction branch.
Example 8. The method of example 5, in which the medical procedure includes using the heart model and thorax model stored in the memory as inputs to a computer-aided or robotic surgery system to facilitate accurate navigation of tools within the patient.
Example 9. The method of example 5, in which the medical procedure includes using the heart model and thorax model stored in the memory to identify a suitable pacing location for inserting a pacemaker lead.
Example 10. The method of any of examples 1-9, in which the processing system performs one or more of the operations using one or more neural network models trained to perform the operations.
Example 11. The method of example 10, further including using machine learning techniques to retrain or refine the one or more neural network models using patient medical imaging data and corresponding electroanatomic heart models and thorax models obtained in subsequent procedures.
Example 12. The method of any of examples 1-10, further including using the adapted thorax model to perform a medical procedure on the patient.
Example 13. A method implemented within a computer processing system for generating and using patient-specific electroanatomic models of a patient's heart and thorax, including: importing medical imaging data of the patient's heart and thorax into the processing system; applying the imported medical imaging data to a neural network model that has been trained to receive heart and thorax medical imaging data as an input and output a patient-specific electroanatomic heart model and a thorax model; and using the patient-specific electroanatomic heart model and thorax model output from the neural network model to perform a medical procedure on the patient.
Example 14. A non-transitory processor-readable medium having stored thereon processor-executable instructions configured to cause a processing system of a computing device to perform operations of the methods of any of examples 1-13.

As used in this application, terminology such as “unit,” “component,” “module,” “system,” etc., is intended to encompass a software-implemented or computer-related entity. These entities may involve, among other possibilities, hardware, firmware, a blend of hardware and software, software alone, or software in an operational state. As examples, a component may encompass a running process on a processor, the processing system itself, an object, an executable file, a thread of execution, a program, or a computing device. To illustrate further, both an application operating on a computing device and the computing device itself may be designated as a component. A component might be situated within a single process or thread of execution or could be distributed across multiple processors or cores. In addition, these components may operate based on various non-volatile computer-readable media that store diverse instructions and/or data structures. Communication between components may take place through local or remote processes, function or procedure calls, electronic signaling, data packet exchanges, and memory interactions, among other known methods of network, computer, processor, or process-related communications.

A number of different types of memories and memory technologies are available or contemplated in the future, any or all of which may be included and used in systems and computing devices that implement the various embodiments. Such memory technologies/types may include non-volatile random-access memories (NVRAM) such as Magnetoresistive RAM (M-RAM), resistive random access memory (ReRAM or RRAM), phase-change random-access memory (PC-RAM, PRAM or PCM), ferroelectric RAM (F-RAM), spin-transfer torque magnetoresistive random-access memory (STT-MRAM), and three-dimensional cross point (3D-XPOINT) memory. Such memory technologies/types may also include non-volatile or read-only memory (ROM) technologies, such as programmable read-only memory (PROM), field programmable read-only memory (FPROM), one-time programmable non-volatile memory (OTP NVM). Such memory technologies/types may further include volatile random-access memory (RAM) technologies, such as dynamic random-access memory (DRAM), double data rate (DDR) synchronous dynamic random-access memory (DDR SDRAM), static random-access memory (SRAM), and pseudo-static random-access memory (PSRAM). Systems and computing devices that implement the various embodiments may also include or use electronic (solid-state) non-volatile computer storage mediums, such as FLASH memory. Each of the above-mentioned memory technologies includes, for example, elements suitable for storing instructions, programs, control signals, and/or data for use in a computing device, system on chip (SOC), or other electronic component. Any references to terminology and/or technical details related to an individual type of memory, interface, or standard memory technology are for illustrative purposes only, and not intended to limit the scope of the claims to a particular memory system or technology unless specifically recited in the claim language.

Various embodiments illustrated and described are provided merely as examples to illustrate various features of the claims. However, features shown and described with respect to any given embodiment are not necessarily limited to the associated embodiment and may be used or combined with other embodiments that are shown and described. Further, the claims are not intended to be limited by any one example embodiment. For example, one or more of the operations of the methods may be substituted for or combined with one or more operations of the methods.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an,” or “the” is not to be construed as limiting the element to the singular.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

In one or more embodiments, the functions described may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include random access memory (RAM), read-only memory (ROM), electrically erasable programmable read-only memory (EEPROM), FLASH memory, solid-state drives (SSD), non-volatile memory express (NVMe) drives, or any other medium that may be used to store target program code in the form of instructions or data structures and that may be accessed by a computer. Modern technologies, such as cloud-based storage solutions, including infrastructure-as-a-service (IaaS) platforms, may offer scalable and distributed options for storing and accessing program code.

In addition, the operations of a method or algorithm may reside as one or any combination or set of codes and/or instructions on a non-transitory processor-readable medium and/or computer-readable medium, which may be incorporated into a computer program product. Emerging technologies, including quantum computing storage media and blockchain-based storage solutions, may further enhance data integrity and security. Artificial intelligence (AI) and machine learning (ML)-optimized hardware accelerators, such as graphical processing systems (GPUs) and tensor processing systems (TPUs), may be used to execute complex algorithms.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. A method implemented within a computer processing system for generating and using patient-specific electroanatomic models of a patient's heart and thorax, comprising:
importing medical imaging data of the patient's heart and thorax into the processing system;
processing the imported medical imaging data by the processing system to segment the heart, blood cavities, and thorax structures to produce segmented images;
processing the segmented images by the processing system to identify anatomical landmarks, including valves, left and right apex, and other relevant landmarks;
verifying by the processing system the alignment of atrial and ventricular heart axes based on the identified anatomical landmarks;
selecting by the processing system a reference heart model from a database of reference electroanatomic heart models
adjusting the reference heart model by the processing system including scaling and modifying the selected reference heart model to match the patient's anatomical landmarks, and storing the resulting adapted heart model in a memory in a format suitable for use in performing a medical procedure on the patient;
selecting a reference thorax model from a database of reference thorax models in a format suitable for use in performing a medical procedure on the patient;
adjusting the selected reference thorax model by the processing system including scaling and modifying the selected reference thorax model to match the patient's anatomical landmarks;
storing the resulting adapted thorax model in the memory in a format suitable for use in performing a medical procedure on the patient; and
using the adapted thorax model to perform a medical procedure on the patient.

2. The method of claim 1, further comprising preprocessing the medical imaging data of the patient's heart and thorax to enhance image quality and consistency using spatial filters, frequency-domain filters, or wavelet-based methods.

3. The method of claim 1, wherein processing the imported medical imaging data by the processing system to segment the heart, blood cavities, and thorax structures to produce segmented images comprises:
generating probability maps by assigning pixels in the medical imaging data to anatomical structures with a highest likelihood of corresponding to such structures; and
creating segmentation masks based on the generated probability maps.

4. The method of claim 1, wherein processing the segmented images by the processing system to identify anatomical landmarks includes the processing system using a trained neural network model to automatically identify the anatomical landmarks.

5. The method of claim 1, further comprising:
repeating operations of:
selecting another reference heart model from the database of reference heart models if there is a reference heart model in the database that has not already been selected;
adapting the selected heart model to match the patient's anatomical landmarks in the memory;
comparing a similarity of the resulting heart model to the patient's heart medical image data with a similarity of a previous adapted heart model saved in the memory; and
storing the resulting heart model in the memory if the resulting heart model is more similar to the patient's heart medical image data than the previous adapted heart model saved in the memory,
wherein using the adapted thorax model to perform a medical procedure on the patient comprises using the heart model and thorax model stored in the memory to perform the medical procedure on the patient.

6. The method of claim 5, wherein comparing the similarity of the resulting heart model to the patient's heart medical image data with the similarity of the previous adapted heart model saved in the memory comprises comparing measures of dimensional similarity of the resulting and previous adapted reference heart models to the patient's anatomical landmarks.

7. The method of claim 5, wherein the medical procedure includes using the heart model and thorax model stored in the memory during an ablation therapy procedure to guide a clinician to an arrhythmia initiation site or conduction branch.

8. The method of claim 5, wherein the medical procedure includes using the heart model and thorax model stored in the memory as inputs to a computer-aided or robotic surgery system to facilitate accurate navigation of tools within the patient.

9. The method of claim 5, wherein the medical procedure includes using the heart model and thorax model stored in the memory to identify a suitable pacing location for inserting a pacemaker lead.

10. The method of claim 1, wherein the processing system performs one or more of the operations using one or more neural network models trained to perform the operations.

11. The method of claim 10, further comprising using machine learning techniques to retrain or refine the one or more neural network models using patient medical imaging data and corresponding electroanatomic heart models and thorax models obtained in subsequent procedures.

12. A method implemented within a computer processing system for generating and using patient-specific electroanatomic models of a patient's heart and thorax, comprising:
importing medical imaging data of the patient's heart and thorax into the processing system;
applying the imported medical imaging data to a neural network model that has been trained to receive heart and thorax medical imaging data as an input and output a patient-specific electroanatomic heart model and a thorax model; and
using the patient-specific electroanatomic heart model and thorax model output from the neural network model to perform a medical procedure on the patient.

13. The method of claim 12, wherein the medical procedure includes using the patient-specific electroanatomic heart model and thorax model stored in the memory during an ablation therapy procedure to guide a clinician to an arrhythmia initiation site or conduction branch.

14. The method of claim 12, wherein the medical procedure includes using the patient-specific electroanatomic heart model and thorax model stored in the memory as inputs to a computer-aided or robotic surgery system to facilitate accurate navigation of tools within the patient.

15. The method of claim 12, wherein the medical procedure includes using the patient-specific electroanatomic heart model and thorax model stored in the memory to identify a suitable pacing location for inserting a pacemaker lead.

16. A computing device, comprising:
a memory; and
a processor system coupled to the memory and configured to perform operations comprising:
importing medical imaging data of the patient's heart and thorax into the processing system;
processing the imported medical imaging data by the processing system to segment the heart, blood cavities, and thorax structures to produce segmented images;
processing the segmented images by the processing system to identify anatomical landmarks including valves, left and right apex, and other relevant landmarks;
verifying by the processing system the alignment of atrial and ventricular heart axes based on the identified anatomical landmarks;
selecting by the processing system a reference heart model from a database of reference electroanatomic heart models;
adjusting the reference heart model by the processing system including scaling and modifying the selected reference heart model to match the patient's anatomical landmarks, and storing the resulting adapted heart model in a memory in a format suitable for use in performing a medical procedure on the patient;
selecting a reference thorax model from a database of reference thorax models in a format suitable for use in performing a medical procedure on the patient;
adjusting the selected reference thorax model by the processing system including scaling and modifying the selected reference thorax model to match the patient's anatomical landmarks;
storing the resulting adapted thorax model in the memory in a format suitable for use in performing a medical procedure on the patient; and
using the adapted thorax model to perform a medical procedure on the patient.

17. The computing device of claim 16, wherein the processing system is configured to perform operations further comprising preprocessing the medical imaging data of the patient's heart and thorax to enhance image quality and consistency using spatial filters, frequency-domain filters, or wavelet-based methods.

18. The computing device of claim 16, wherein the processing system is configured to perform operations such that processing the imported medical imaging data by the processing system to segment the heart, blood cavities, and thorax structures to produce segmented images comprises:
generating probability maps by assigning pixels in the medical imaging data to anatomical structures with a highest likelihood of corresponding to such structures; and
creating segmentation masks based on the generated probability maps.

19. The computing device of claim 16, wherein the processing system is configured to perform operations such that processing the segmented images by the processing system to identify anatomical landmarks includes the processing system using a trained neural network model to automatically identify the anatomical landmarks.

20. The computing device of claim 16, wherein the processing system is configured to perform operations further comprising:
repeating operations of:
selecting another reference heart model from the database of reference heart models if there is a reference heart model in the database that has not already been selected;
adapting the selected heart model to match the patient's anatomical landmarks in the memory;
comparing a similarity of the resulting heart model to the patient's heart medical image data with a similarity of a previous adapted heart model saved in the memory; and
storing the resulting heart model in the memory if the resulting heart model is more similar to the patient's heart medical image data than the previous adapted heart model saved in the memory,
wherein using the adapted thorax model to perform a medical procedure on the patient comprises using the heart model and thorax model stored in the memory to perform the medical procedure on the patient.

21. The computing device of claim 16, wherein the processing system is configured to perform operations such that comparing the similarity of the resulting heart model to the patient's heart medical image data with the similarity of the previous adapted heart model saved in the memory comprises comparing measures of dimensional similarity of the resulting and previous adapted reference heart models to the patient's anatomical landmarks.

22. The computing device of claim 16, wherein the processing system is configured to perform operations such that the medical procedure includes using the heart model and thorax model stored in the memory during an ablation therapy procedure to guide a clinician to an arrhythmia initiation site or conduction branch.

23. The computing device of claim 16, wherein the processing system is configured to perform operations such that the medical procedure includes using the heart model and thorax model stored in the memory as inputs to a computer-aided or robotic surgery system to facilitate accurate navigation of tools within the patient.

24. The computing device of claim 16, wherein the processing system is configured to perform operations such that the medical procedure includes using the heart model and thorax model stored in the memory to identify a suitable pacing location for inserting a pacemaker lead.

25. The computing device of claim 16, wherein the processing system is configured to perform operations such that the processing system performs one or more of the operations using one or more neural network models trained to perform the operations.

26. The computing device of claim 16, wherein the processing system is configured to perform operations further comprising using machine learning techniques to retrain or refine the one or more neural network models using patient medical imaging data and corresponding electroanatomic heart models and thorax models obtained in subsequent procedures.

27. A computing device, comprising:
a memory; and
a processor system coupled to the memory, including a neural network model that has been trained to receive heart and thorax medical imaging data as an input and output a patient-specific electroanatomic heart model and a thorax model, and configured with executable instructions to perform operations comprising:
importing medical imaging data of a patient's heart and thorax;
applying the imported medical imaging data to neural network model; and
using the patient-specific electroanatomic heart model and thorax model output from the neural network model to perform a medical procedure on the patient.

28. The computing device of claim 27, wherein the medical procedure includes using the patient-specific electroanatomic heart model and thorax model stored in the memory during an ablation therapy procedure to guide a clinician to an arrhythmia initiation site or conduction branch.

29. The computing device of claim 27, wherein the medical procedure includes using the patient-specific electroanatomic heart model and thorax model stored in the memory as inputs to a computer-aided or robotic surgery system to facilitate accurate navigation of tools within the patient.

30. The computing device of claim 27, wherein the medical procedure includes using the patient-specific electroanatomic heart model and thorax model stored in the memory to identify a suitable pacing location for inserting a pacemaker lead.
